(19) United States
(12) Patent Application Publication
(10) Pub. No.: US 2023/0117826 A1
(43) Pub. Date: Apr. 20, 2023

(54) COMPOUNDS AND COMPOSITIONS FOR RETINAL INJURY DETECTION AND METHODS OF USING SAME

(71) Applicant: MOLECULAR TARGETING TECHNOLOGIES, INC., West Chester, PA (US)

(72) Inventors: Brian D. GRAY, Exton, PA (US); Silvia C. FINNEMANN, South Salem, NY (US); Francesca MAZZONI, Bronx, NY (US); Koon Y. PAK, Malvern, PA (US)

(21) Appl. No.: 17/759,912

(22) PCT Filed: Feb. 5, 2020
(86) PCT No.: PCT/US20/16846

§ 371 Date: Aug. 1, 2022

(51) Int. Cl. C07D 401/14; C07D 491/04; A61P 27/02; A61K 49/00

(52) U.S. Cl. CPC C07D 401/14; C07D 491/04; A61P 27/02; A61K 49/005

(57) ABSTRACT

Described are compounds, compositions, and methods suitable for diagnosing individuals with eye injuries and/or diseases. The compounds of the present disclosure have fluorescent groups and bis-dipicolylamine groups, which may be substituted or unsubstituted. The fluorescent group and bis-dipicolylamine group are connected by linking groups. The compositions may be formulated and administered as an eye drop. The methods may be used to track and/or label dying cells associated with eye injuries and/or diseases, such as, for example, retinal degenerations including, but not limited to, retinitis pigmentosa, glaucoma, diabetic retinopathy, and age-related macular degeneration.

### STATEMENT REGARDING FEDERALLY SPONSORED RESEARCH

This invention was made with government support under contract no. EY26215 awarded by the National Institutes of Health. The government has certain rights in the invention.

### BACKGROUND OF THE DISCLOSURE

Detection of apoptosis in retinal degenerations is of critical importance in diagnosis, treatment, and monitoring of these debilitating diseases. Apoptosis is a regulated form of programmed cell death that plays an essential role in numerous physiological processes and diseases including hereditary and induced forms of retinal degeneration. During early apoptosis, enzymatic translocation of anionic phosphatidylserine (PS) from the inner to the outer leaflet of the plasma membrane serves as an “eat me” signal, which triggers clearance phagocytosis of apoptotic cells.

Two fluorescent dyes current approved for clinical use in ophthamology are fluorescein and indocyanine green, these dyes are used for angiography and have fluorescence ex/em properties of 488/510 nm and 780/795 nm respectively. The devices for detection of these dyes are widely available in hospitals and clinics.

However, previously used dyes are administered through intravitreal injection and require DMSO, which is known to cause retinal apoptosis at concentrations ≥1% v/v.

Thus, there is a need for compositions and methods to detect retinal degeneration without using DMSO and intravitreal injection.

### SUMMARY OF THE DISCLOSURE

Disclosed are compounds and compositions (e.g., eye drop compositions) suitable for detection of PS-exposing apoptotic photoreceptors. The compounds and compositions may be suitable for labelling and tracking dying cells without intraocular injection. It is believed the ability to track and label dying cells will change the diagnosis of eye injuries/diseases (e.g., retinal degenerations such as, for example, retinitis pigmentosa, glaucoma, diabetic retinopathy, and age-related macular degeneration) because such eye injuries/diseases may be diagnosed at earlier stages than the current standard of care. Dying cells may be tracked in vivo over time using non-invasive imaging without the necessity of intraocular injection.

The present disclosure provides compounds that bind to apoptotic photoreceptors in the eye. Also provided are compositions comprising the compounds and methods of using the compounds and/or compositions.

In an aspect, the present disclosure provides compounds comprising fluorescent groups and bis-dipicolylamine groups, which may be substituted or unsubstituted. The fluorescent group and bis-dipicolylamine are connected by linking groups. The bis-dipicolylamine groups bind to phosphatidylserine, which is externalized during apoptosis.

In an aspect, the present disclosure provides compositions comprising one or more compounds of the present disclosure. The compositions may comprise one or more pharmaceutically acceptable carriers.

In various examples, one or more compounds of this disclosure can be provided in the form of eye drops. In various examples, the compounds are present in typical eye drop volumes, and are used by administering 1-2 drops/eye at approximately 0.05 to 0.1 mL per eye, including every 0.01 mL value and range therebetween.

In an aspect, the present disclosure provides methods of using one or more compounds of the present disclosure. For example, the compounds can be used to diagnose any applicable ophthalmic condition and/or disease, including, for example, back of the eye diseases, which involve markers that define apoptosis and any related or associated pathway involved in the disease process. A method of diagnosing comprises administering to an individual one or more compounds of the present disclosure or a composition comprising one or more compounds of the present disclosure. In various examples, a composition comprises one or more compounds of the present disclosure.

A method of the present disclosure comprises determining if there is and/or the amount of retinal apoptosis in an individual in need of treatment. Methods of the present disclosure do not involve administration via intraocular injection.

Methods of the present disclosure may be performed in vivo on a subject in need of treatment having or suspected of having an eye disease and/or eye injury.

In an aspect, the present compounds and compositions may be used as research tools.

### BRIEF DESCRIPTION OF THE FIGURES

For a fuller understanding of the nature and objects of the disclosure, reference should be made to the following detailed description taken in conjunction with the accompanying figures.

FIG. 1 shows comparison of staining of apoptotic photoreceptors by fluorescent PS probes Compound II and pSIVA applied as eye drop, by intravitreal injection, or to retina ex vivo. (a) Experimental paradigm for eye drop probe application. (b and c) Representative maximal projection images of live dissected retina or RPE, as indicated, of Compound II (b) or pSIVA (c) staining of p25 RCS or WT rat tissues as indicated. Tissues of equal volumes were imaged live immediately following dissection 24 hours after eye drop application as in a. Scale bar, 50 μm. (d) Representative images of live dissected retina after Compound II or pSIVA intravitreal injection. Images from control eyes injected with HBSS buffer are also shown to the right of each fluorescent probe, as indicated. Scale bar, 20 μm. (e) Representative live imaging of co-staining by Compound II and pSIVA probes following application of mixed probes to freshly dissected RCS rat retina ex vivo. Scale bar, 10 μm. All retina flatmounts are shown photoreceptor side up.

FIG. 2 shows live imaging of apoptotic photoreceptors in vivo by whole animal scanning. (a) Representative whole animal scans of p25 RCS and WT rats 24 hours after Compound II or HBSS buffer eye drop application as indicated. Intensity range on top shows false color scale. Encircled regions show quantified areas. (b) Quantification of fluorescence intensity as in a of p25 rats 24 and 72 hours after Compound II application; n=7 animals per group. (c) Quantification of fluorescence intensity 24 hours after eye drops of RCS rats treated with Compound II or HBSS eye drops at p16 (16) with repeat at p23 (23r, black bar) side by side with p23 siblings that had not been treated before (23, white bar); n=6 animals per group. (d) Quantification of fluorescence intensity 24 hours after eye drops of RCS rats at p18, p25, and p60 as indicated; n=5 animals per group. (e) Quantification of fluorescence intensity 24 and 72 hours after Compound II application in pigmented mertk^{-/-} mice; n=9 animals per group. (f) Quantification of fluorescence intensity 24 and 72 hours after Compound II application in LD rats; n=7 animals per group. (b-f) All bars show mean±SEM. All asterisks indicate P<0.05 by ANOVA and Tukey post-hoc test. n. s. indicates difference not significant.

FIG. 3 shows live imaging of apoptotic photoreceptors in vivo by retinal imaging. (a) Representative fluorescence images of photoreceptors and fundus images of p25 RCS and WT rats 24 hours after Compound II or HBSS solvent eye drop application as indicated. (b) Quantification of fluorescence intensity after background subtraction in areas indicated by dashed lines. *, P<0.05 by Student's t-test. (c) Photoreceptor fluorescence and (d) quantification as in b of fluorescence intensity maxima. *, P<0.05 Student's t-test. (e) Photoreceptor fluorescence and (f) quantification of areas of fluorescent intensities specific to retinal quadrants. *, P<0.05; two-way ANOVA and Tukey post-hoc test. (b, d, f) All bars show mean±SEM, n=3 animals per group.

FIG. 4 shows largely intact RCS rat retina at p25 indicative of early stage retinal degeneration. Representative light micrographs of central and peripheral areas from H&E-stained retina sections of p25 SD WT and RCS rats. Representative images of central (a) and peripheral (b) areas in WT and RCS retinal/RPE tissue, respectively. Note persistence but abnormal appearance of photoreceptor inner and outer segments in RCS eye. os, photoreceptor outer segments; is, photoreceptor inner segments; onl, outer nuclear layer; opl, outer plexiform layer; inl, inner nuclear layer; ipl, inner plexiform layer; gcl, ganglion cell layer. Scale bar, 50 μm.

FIG. 5 shows penetration of Compound II administered as eye drop into the rat eye irrespective of retinal degeneration. Quantification of Compound II in HBSS buffer applied to the exterior of enucleated eyeballs (white bars) or of internal fluid retrieved from eyecups following extraction of the lens (black bars). Eyeballs were harvested 3 hours after Compound II eye drop application of p25 WT and RCS rats as indicated. Bars show mean SEM, n=3 animals per group; Compound II levels in the same compartment of WT and RCS rat eyes did not differ significantly as per two-way ANOVA.

FIG. 6 shows lack of direct toxicity or adverse effects on vision of Compound II eye drops. (a) Scotopic electroretinogram (ERG) recordings from littermate RCS rats 72 hours after Compound II or HBSS eye drops. ERG curves show averaged responses from one representative animal each. (b) a-wave and (c) b-wave amplitudes of ERGs as in a. White bars: Compound II eye drops; black bars: HBSS solvent eye drops; mean±SEM; n=4 rats per group; differences not significant by 2-way ANOVA. Bars show one representative ERG experiment. The experiment was performed 4 times with identical results, each time testing 3-4 rats per group.

FIG. 7 shows characterization of RCS rat retina at ages prior to photoreceptor apoptosis (p18) and following complete photoreceptor loss (p60). Representative light micrographs of central areas from H&E-stained retina sections of (a) p18, (b) p60 RCS rats and (c) p60 WT rats, as indicated. Note absence of outer nuclei layer of photoreceptor cell nuclei in p60 RCS rat retina. onl, outer nuclear layer; inl, inner nuclear layer; gcl, ganglion cell layer. Scale bar, 50 m. (d) Representative immunoblot of whole eye lysates of RCS and WT rats sacrificed at ages as indicated by numbers above blots (p18, p25, p60, p23 and siblings p23* that had received eye drops at P16). The same blot membrane was probed as indicated for caspase-3 whose cleavage is indicative of apoptosis, tubulin as universal cell marker, and PSD95 as synapse marker. Note increase of cleaved, active caspase-3 indicative of ongoing apoptosis between p23 and p25 but similar levels of PSD95 in RCS retina at p18 to p25 as in WT retina. Also note very little apoptosis in RCS retina at p18 and none at p60, at which age synapses are diminished. The experiment was repeated three times with identical results.

FIG. 8 shows early stage retinal degeneration in pigmented mertk^{-/-} mouse retina at p28 and photoreceptor loss by p60. Images show representative central areas of the retina of p18 WT and mertk^{-/-} mice at ages as indicated. Nuclear staining with rhodopsin was performed. Staining indicative of photoreceptor rod outer segments is shown. p18 WT shows normal retinal morphology and tissue organization. Note outer nuclei layer diminishes by p60 indicating photoreceptor cell death. os, photoreceptor outer segments; is, photoreceptor inner segments; onl, outer nuclear layer; opl, outer plexiform layer; inl, inner nuclear layer; ipl, inner plexiform layer; gcl, ganglion cell layer. Scale bar, 50 μm.

FIG. 9 shows induction of early stage retinal degeneration in p24 SD WT albino rats by light damage (LD). (a) Experimental paradigm. Bar indicates animals were maintained in darkness for three days before bright light (LD) or room light (control) exposure at p24 for 24 hours. Rats were kept in the dark for the remainder of the experiment. Four days after LD, Compound II eye drops were applied. 24 hours and 72 hours later, animals were subjected to live imaging, indicated by arrowheads. (b) Map of retinal regions. (c) Representative images of ventral and dorsal regions (as in b) as indicated of retina from LD rats sacrificed 5 days after LD (upper panels) and from control rats (ctrl) (lower panels). Rhodopsin staining and nuclei staining are shown. Scale bar, 50 μm. RPE, retinal pigment epithelium; ipl, inner plexiform layer; opl, outer plexiform layer; gcl, ganglion cell layer. Note thinning outer segment layer in ventral LD rat retina (1, 2) but severe inner and outer segment disruption and rhodopsin mis-localization to outer nuclear layer in dorsal regions (3, 4) as expected. Insets show close-up of the RPE with opsin-positive phagosomes confirming that RPE in LD rats maintains clearance phagocytosis activity like control RPE. Scale bar inset, 10 μm.

FIG. 10 shows uncut original immunoblots used to compile panels for FIG. 7d.

FIG. 11 shows synthetic routes for water-soluble 488 nm excitation dyes. Reagents: (i) DPA-amine, DMF; (ii) 2Zn(NO₃⁻)₂, MeOH; (iii) Et₃N, CH₂Cl₂, reflux; (iv) DCC, NHS, DMF; (v) 2,4-dimethylpyrrole, CH₂Cl₂, TFA; (vi) p-chloranil, CH₂Cl₂; (vii) BF₃(OEt)₂.

FIG. 12 shows synthetic routes for water soluble 780 nm excitation dyes. Reagents: (i) N-[(3-(Anilinomethylene)-2-chloro-1-cyclohexen-1-yl)methylene]aniline monohydrochloride, NaOAc, EtOH, heat; (ii) 4-carboxyphenylboronic acid, Pd(PPh₃)₄, heat; (iii) DCC, NHS, DMF; (iv) DPA-amine, DMF; (v) 2Zn(NO₃⁻)₂, MeOH; (vi) N-[5-(phenylamino)-2,4-pentadienylidene] aniline monochloride, Ac₂O, heat.

FIG. 13 shows a synthetic scheme of a compound of the present disclosure.

FIG. 14 shows live imaging of apoptotic photoreceptors in vivo by retinal imaging photoreceptor fluorescence with Compound II.

FIG. 15 shows in vivo quantification of compounds of the present disclosure. (A) shows Compound V and (B) shows ratio of Compound (8) to Compound I.

FIG. 16 shows ex vivo retina images, 3 hours (h) after application of eye drops comprising compounds of the present disclosure.

FIG. 17 shows compounds of the present disclosure.

FIG. 18 shows rats received eye drops with compounds I or (8) as indicated 3 hours before sacrifice. Freshly dissected retinas were mounted photoreceptor side up and immediately imaged live to detect compound fluorescence labeling of apoptotic photoreceptor neurons. Representative maximal projections of x-y image stacks are shown. Scale bars: 25 μm.

FIG. 19 shows rats received eye drops with compounds II or (22) as indicated 3 hours before sacrifice. Freshly dissected retinas were mounted photoreceptor side up and immediately imaged live to detect compound fluorescence labeling of apoptotic photoreceptor neurons. Representative maximal projections of x-y image stacks are shown. Scale bars: 25 μm.

FIG. 20 shows rats received eye drops with compounds III or IV as indicated 24 hours before sacrifice. Freshly dissected retinas were mounted photoreceptor side up and immediately imaged live to detect compound fluorescence labeling of apoptotic photoreceptor neurons. Representative maximal projections of x-y image stacks are shown. Scale bars: 25 μm.

FIG. 21 shows rats received eye drops with compound V 3 hours before sacrifice. Freshly dissected retinas were mounted photoreceptor side up and immediately imaged live to detect compound fluorescence labeling of apoptotic photoreceptor neurons. Representative maximal projections of x-y image stacks are shown. Scale bars: 25 μm.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

All ranges provided herein include all values that fall within the ranges to the tenth decimal place, unless indicated otherwise.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include:

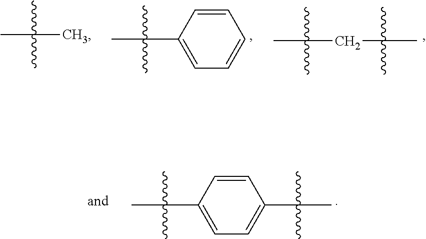

As used herein, unless otherwise indicated, the term “alkyl” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₃₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). The alkyl group may be unsubstituted or substituted with one or more substituents. Examples of substituents include, but are not limited to, substituents such as, for example, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, unless otherwise indicated, the term “aliphatic” refers to branched or unbranched hydrocarbon groups that, optionally, contain one or more degrees of unsaturation. Degrees of unsaturation can arise from, but are not limited to, aryl groups and cyclic aliphatic groups. For example, the aliphatic groups/moieties are a C₁ to C₃₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). The aliphatic group can be unsubstituted or substituted with one or more substituent. Examples of substituents include, but are not limited to, substituents such as, for example, halogens (—F, —Cl, —Br, and —I), aliphatic groups (e.g., alkyl groups and the like), halogenated aliphatic groups (e.g., trifluoromethyl group), aryl groups, halogenated aryl groups, substituted amine groups, carboxylic acids groups, protected alcohol groups, ether groups, ester groups, thioether groups, thioester groups, substituted carbamate groups, substituted amide groups, alkenes with a long alkyl chain between connecting it to the epoxide, and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, the term “heteroaryl” refers to a C₁ to C₁₄ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, and C₁₄). The heteroaryl groups may be substituted or unsubstituted. Examples of heteroaromatic groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, substituents such as, for example, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof.

Disclosed are compounds, compositions (e.g., eye drop compositions), and methods suitable for detection of PS-exposing apoptotic photoreceptors. The compounds and compositions may be suitable for diagnosing through, for example, labelling and tracking dying cells without intraocular injection. It is believed the ability to track and label dying cells will change the diagnosis of eye injuries and/or diseases (e.g., retinal degenerations such as, for example, retinitis pigmentosa, glaucoma, diabetic retinopathy, and age-related macular degeneration) because such eye injuries/diseases may be diagnosed at earlier stages than the current standard of care. Dying cells may be tracked in vivo over time using non-invasive imaging without the necessity of intraocular injection.

The present disclosure provides compounds that bind to apoptotic photoreceptors in the eye. Also provided are compositions comprising the compounds and methods of using the compounds and/or compositions.

In an aspect, the present disclosure provides compounds comprising fluorescent groups and bis-dipicolylamine groups, which may be substituted or unsubstituted. The fluorescent group and bis-dipicolylamine group are connected by linking groups. The bis-dipicolylamine groups bind to phosphatidylserine (PS), which is externalized during apoptosis.

In various examples, a compound of the present disclosure has the following structure:

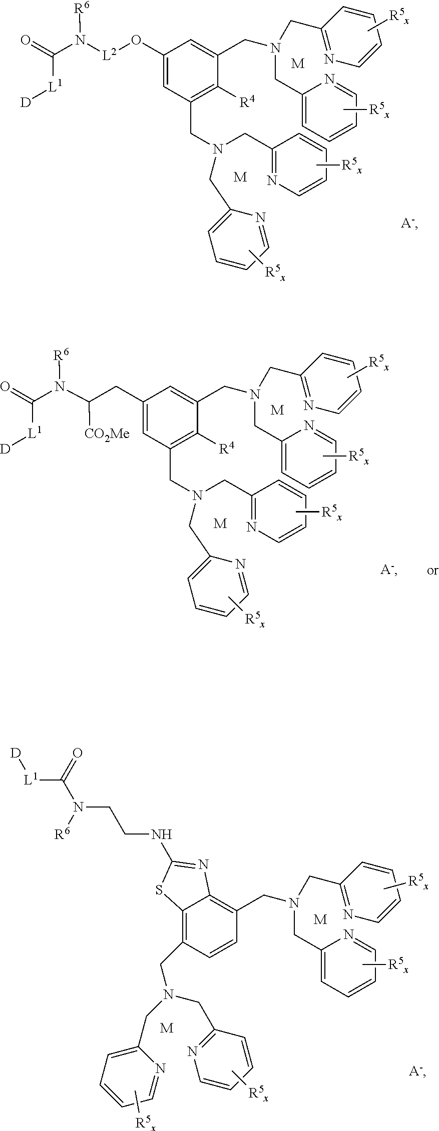

where D is chosen from:

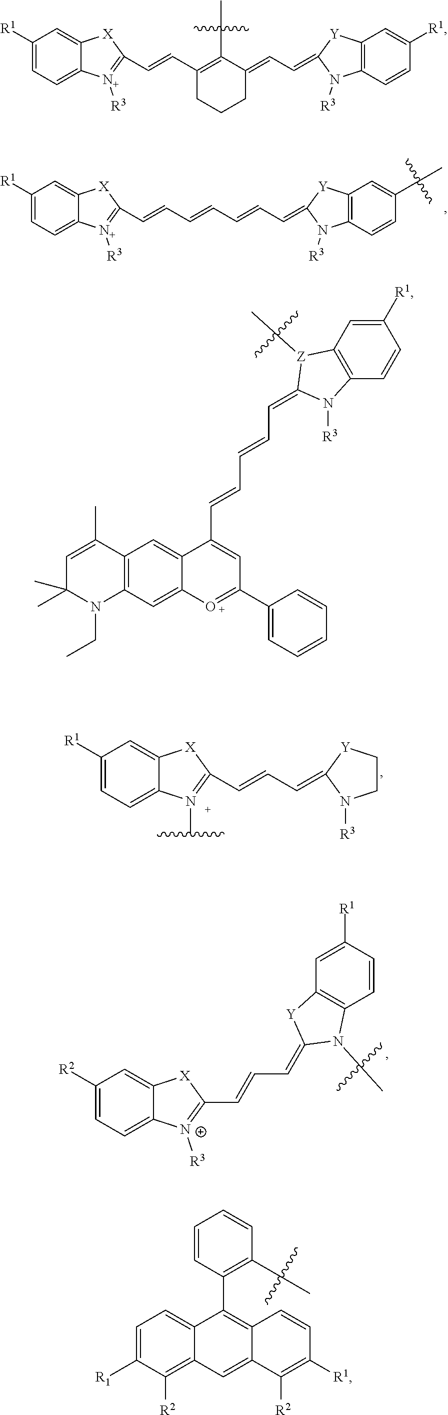

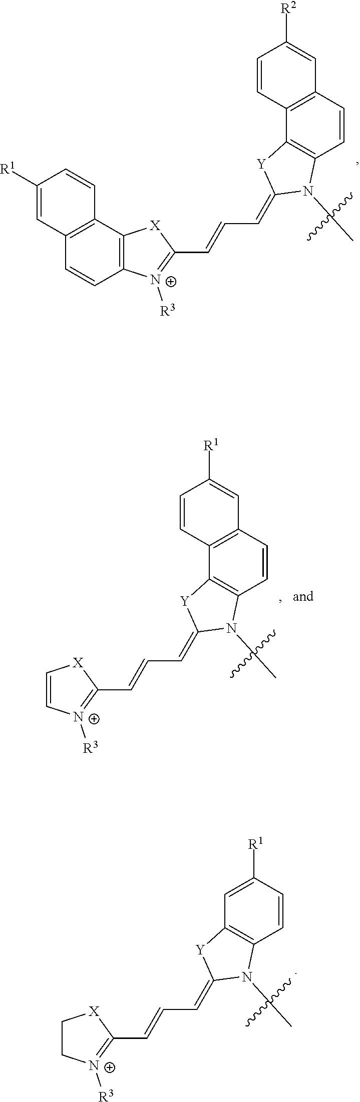

R¹ and R² are polar groups that may be the same or different. R³ is chosen from alkyl groups, alkyl groups, polyethylene glycol (PEG) groups (e.g., the PEG groups have 1-12 ethylene glycol repeat unit), and C₁ to C₁₂ alkyl sulfonic acid groups. R⁴ is chosen from —H and —OH. R⁵ is chosen from —H, alkyl groups (e.g., methyl, ethyl, and the like), amine groups (e.g., primary amines, secondary amines, and tertiary amines), amide groups, carbamide groups, carbamate groups, and halogen groups. R⁶ is —H or an alkyl group (e.g., methyl, ethyl, propyl, butyl and the like). L¹ and L² are independently optional and are linking groups that may be the same or different. A is one or more counterions. M is a divalent cation. x is individually at each occurrence 1-4. X and Y are independently C(R⁶)₂, O or S, where each R⁶ is the same or different. Z is C(R₆). Non-limiting examples of carbamide groups include:

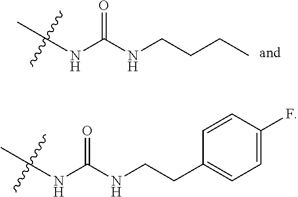

The compounds of the present disclosure may be water soluble or substantially water soluble. That is, a compound of the present disclosure may dissolve in an aqueous solution having ≤20% DMSO v/v, ≤19% DMSO v/v, ≤18% DMSO v/v, ≤17% DMSO v/v, ≤16% DMSO v/v, ≤15% DMSO v/v, ≤14% DMSO v/v, ≤13% DMSO v/v, ≤12% DMSO v/v, ≤11% DMSO v/v, ≤10% DMSO v/v, ≤9% DMSO v/v, ≤8% DMSO v/v, ≤7% DMSO v/v, ≤6% DMSO v/v, ≤5% DMSO v/v, ≤4% DMSO v/v, ≤3% DMSO v/v, ≤2% DMSO v/v, ≤1% DMSO v/v, K 0.9% DMSO v/v, K 0.8% DMSO v/v, ≤0.8% DMSO v/v, ≤0.7% DMSO v/v, ≤0.6% DMSO v/v, ≤0.5% DMSO v/v, ≤0.4% DMSO v/v, ≤0.3% DMSO v/v, ≤0.2% DMSO v/v, K 0.1% DMSO v/v, or no DMSO. In various examples, no DMSO is required to dissolve a compound of the present disclosure in an aqueous solution (e.g., water, saline, or a buffered solution) or no DMSO is present in aqueous solution.

Polar groups are groups that are hydrophilic and/or have a charge. Non-limiting examples of polar groups include sulfonic acid groups (e.g., protonated and/or deprotonated sulfonic acid groups, including sodium or potassium salts thereof), C₁ to C₁₂ alkyl sulfonic acid groups, PEG groups (e.g., the PEG groups have 1-12 ethylene glycol repeat unit), sugar groups (e.g., D and L monosaccharides, including, but not limited to pentoses (e.g., aldopentoses, ketopentoses, and the like), such as, for example, arabinose groups, lyxose groups, ribose groups, xylose groups, ribulose groups, xylulose groups, and the like; hexoses (e.g., aldohexoses, ketohexoses, and the like), such as for example, allose groups, altrose groups, glucose groups, mannose groups, gulose groups, idose groups, galactose groups, talose groups, psicose groups, fructose groups, sorbose groups, tagatose groups, and the like; and the like; disaccharides, such as, for example, sucrose groups, lactulose groups, lactose groups, maltose groups, trehalose groups, cellobiose groups, and chitobiose groups, and the like; and polysaccharides having up to 12 saccharide groups), amine groups (such as, for example, ammonium groups (e.g., —NH₃⁺, —NH₂R, —NHRR′, where R and R′ are alkyl groups that may be the same or different, such as, for example —NMe₃⁺)), phosphonic acid groups, and combinations thereof. When polar groups are ionized, they may have various counterions (e.g., sodium ions, potassium ions, and the like). Ionized compounds may be pharmaceutically acceptable salts.

Linking groups may be any suitable linking group. Linking groups may be chosen from aryl groups, C₂ to C₆ aliphatic groups, and polyethylene glycol groups. Non-limiting examples of linking groups include, alkyl groups (e.g., methyl groups, ethyl groups, propyl groups, butyl groups, pentyl groups, hexyl groups, and the like), PEG groups having 2-6 ethylene glycol repeat units, aryl groups (e.g., phenyl groups, and the like) and the like, and combinations thereof.

A divalent cation may be chelated to a tertiary amine and one or more heteroaryl groups. Examples of divalent cations include, but are not limited to, Mn²⁺, Fe²⁺, Co²⁺, Ni²⁺, Cu²⁺, Zn²⁺, and the like, and combinations thereof.

Pyridinyl groups of the compound may have various one or more substituents that are the same or different. Each pyridinyl group may have 1-4 substituents. Non-limiting examples of substituents include hydrogen, halogens (—F, —Cl, —Br, and —I), aliphatic groups (e.g., alkyl groups, alkenyl groups, alkynyl groups, and the like), aryl groups, alkoxide groups, amine groups, amide groups, carbamide groups, carbamate groups, carboxylate groups, carboxylic acids, ether groups, alcohol groups, alkyne groups (e.g., acetylenyl groups and the like), and the like, and combinations thereof. In various examples, the pyridinyl groups are unsubstituted (i.e., the pyridinyl groups have four hydrogen atoms).

In various examples, a compound of the present disclosure has the following structure:

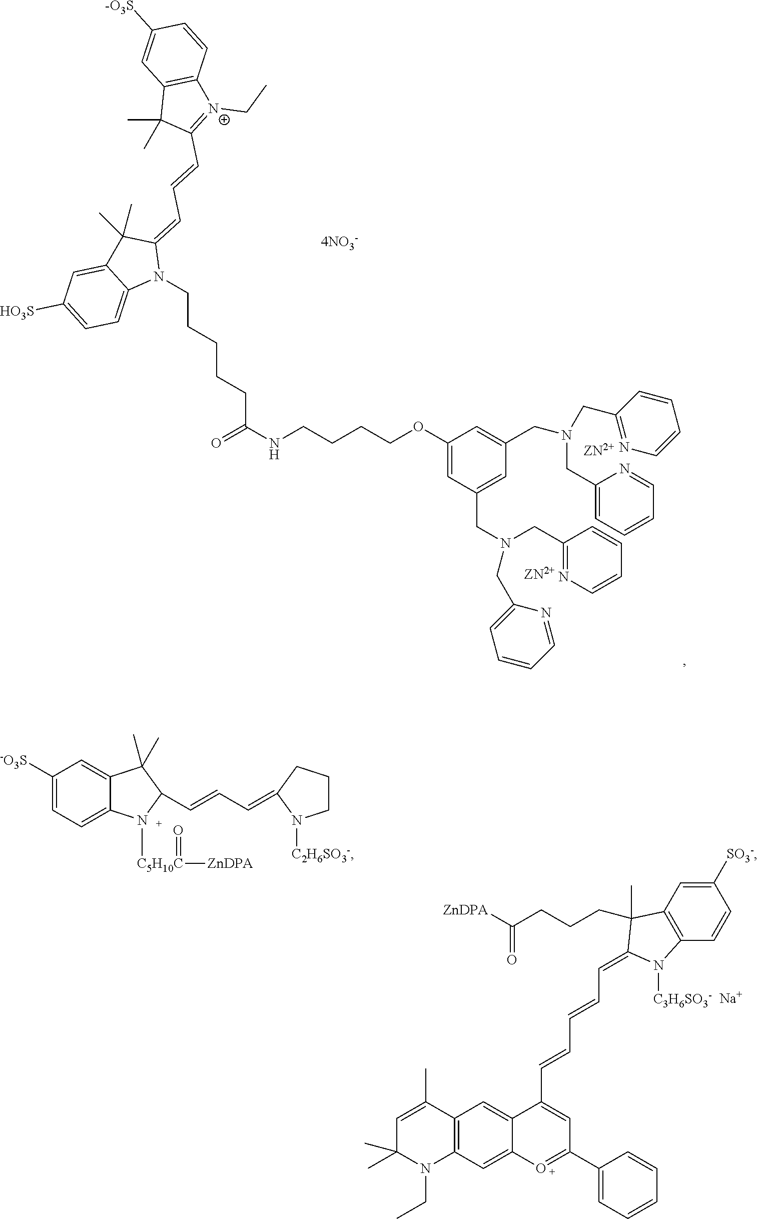

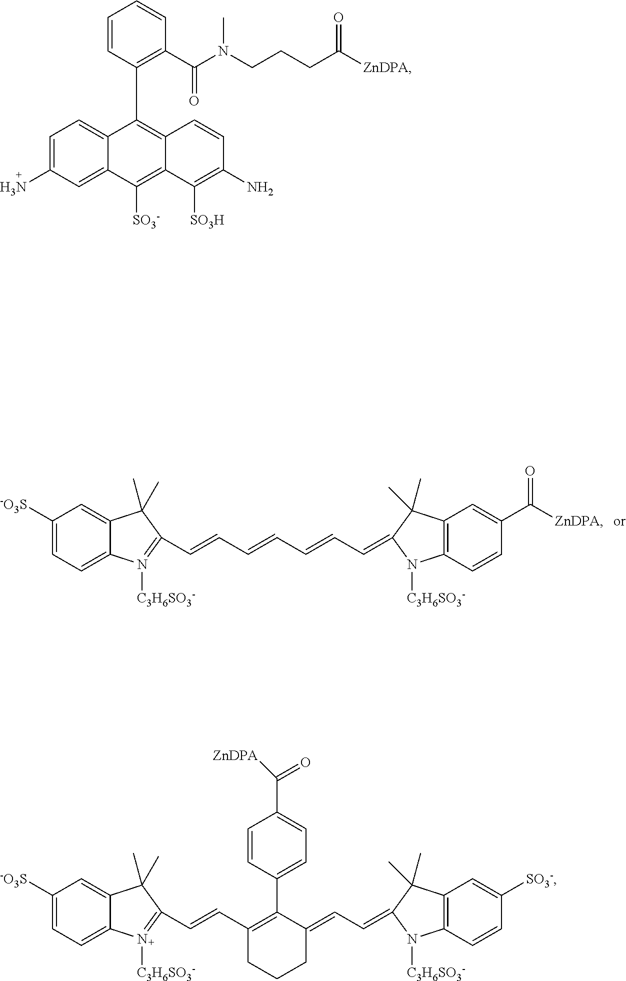

where ZnDPA has the following structure:

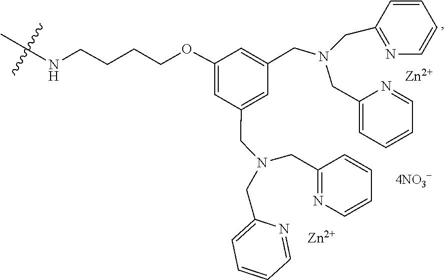 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

where the alkyl sulfonic acid groups (e.g., CₓH_{y}SO₃⁻ and CₓH_{y}SO₃H) may be linear alkyl groups (e.g., n-propyl, n-pentyl).

In an aspect, the present disclosure provides compositions comprising one or more compounds of the present disclosure. The compositions may comprise one or more pharmaceutically acceptable carriers.

The compositions described herein may include one or more standard pharmaceutically acceptable carriers. Pharmaceutically acceptable carriers may be determined in part by the particular composition being administered, as well as by the particular method used to administer the composition. Accordingly, there are a wide variety of suitable formulations of pharmaceutical compositions of the present disclosure. The compounds may be freely suspended in a pharmaceutically acceptable carrier or the compounds may be encapsulated in liposomes and then suspended in a pharmaceutically acceptable carrier. Examples of carriers include solutions, suspensions, emulsions, solid injectable compositions that are dissolved or suspended in a solvent before use, and the like. The compositions may be prepared by dissolving, suspending, or emulsifying one or more of the active ingredients (e.g., a compound of the present disclosure) in a diluent. Examples of diluents, include, but are not limited to, distilled water, physiological saline, vegetable oil, alcohol, dimethyl sulfoxide, and combinations thereof. Further, the injections may contain stabilizers, solubilizers, suspending agents, emulsifiers, soothing agents, buffers, preservatives, and the like. The injections may be sterilized in the final formulation step or prepared by sterile procedure. The composition of the disclosure may also be formulated into a sterile solid preparation, for example, by freeze-drying, and can be used after sterilized or dissolved in sterile injectable water or other sterile diluent(s) immediately before use. Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, Pa. Lippincott Williams & Wilkins. Effective formulations are formulated for topical application to the eye.

The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

In various examples, one or more compounds of this disclosure can be provided in the form of eye drops. The eye drops may comprise one or more of steroids, antihistamines, sympathomimetics, beta receptor blockers, parasympathomimetics, parasympatholytics, prostaglandins, nonsteroidal anti-inflammatory drugs (NSAIDs), antibiotics, antifungal, topical anesthetics, or the like, or a combination thereof. The eye drops may be for use with any eye condition. In various examples, the eye drops comprise artificial tears. The eye drops may be free of medications, other than the presently described compounds. In various examples, an eye drop comprises a compound of the present disclosure and water or saline. In various examples, the compounds are present in typical eye drop volumes, and are used by administering 1-2 drops/eye at approximately 0.05 to 0.1 mL per eye, including every 0.01 mL value and range therebetween.

In an aspect, the present disclosure provides methods of using one or more compounds of the present disclosure. For example, the compounds can be used to diagnose any applicable ophthalmic condition and/or disease, including, for example, back of the eye diseases, which involve markers that define apoptosis and any related or associated pathway involved in the disease process. A method of treating comprises administering to an individual one or more compounds of the present disclosure or a composition comprising one or more compounds of the present disclosure. In various examples, a composition comprises one or more compounds of the present disclosure.

In various examples, a composition comprising one or more compounds described herein is used to diagnose an eye disorder that comprises one or more diseases and/or injuries to the retina. Non-limiting examples of diseases include age-related macular degeneration (AMD) and retinal degeneration (RD), such as, for example, photoreceptor degeneration(s), such as, for example, retinitis pigmentosa (RP) and diabetic retinopathy (DR). In an example, the individual has dry, atrophic (nonexudative) age-related macular degeneration, defined as progressive age-related degeneration of the macular associated with retinal pigment epithelial changes including atrophy and drusen, which is a common cause of vision loss in adults. In various examples, the disorder comprises one or more diseases or injury to the cornea. In various examples, the individual has glaucoma, which may include primary, secondary and/or congenital glaucoma.

The back of the eye diseases can deal with cellular or subcellular components of the back of the eye anatomy and histology including the retina and all of the 10 or more cells comprising the layers of the retina (e.g., inner limiting membrane, retinal ganglion cell layer, inner plexiform layer, inner nuclear layer, outer plexiform layer, photoreceptor layer, outer limiting membrane, inner segment, outer segment, retinal pigment epithelium, Bruck's membrane) and other structures including vitreous and choroid. Additional components of the back of the eye include the ciliary body, iris, uvea and the retinal pigment cells. Back of the eye diseases include processes that involve the optic nerve and all of its cellular and subcellular components such as the axons and their innervations. These include disease such as primary open angle glaucoma, acute and chronic closed angle glaucoma and any other secondary glaucoma. Diseases of the back of the eye also may include myopic retinopathies, macular edema such as clinical macular edema or angiographic cystoid macular edema arising from various etiologies such as diabetes, exudative macular degeneration and macular edema arising from laser treatment of the retina, diabetic retinopathy, age-related macular degeneration, retinopathy of prematurity, retinal ischemia and choroidal neovascularization and like diseases of the retina; genetic disease of the retina and macular (e.g., wet and dry macular degeneration); pars planitis; Posner Schlossman syndrome; Bechet's disease; Vogt-Koyanagi-Harada syndrome; hypersensitivity reactions; toxoplasmosis chorioretinitis; inflammatory pseudo-tumor of the orbit; chemosis; conjunctival venous congestion; periorbital cellulitis; acute dacryocystitis; nonspecific vasculitis; sarcoidosis and cytomegalovirus infection.

A method of the present disclosure comprises determining if there is (e.g., the presence of) and/or the amount of retinal apoptosis in an individual in need of treatment. A method of the present disclosure comprises: administering to an eye of the individual an effective amount of a first composition of the present disclosure; a second composition comprising a pharmaceutically acceptable carrier and one or more of the following compounds:

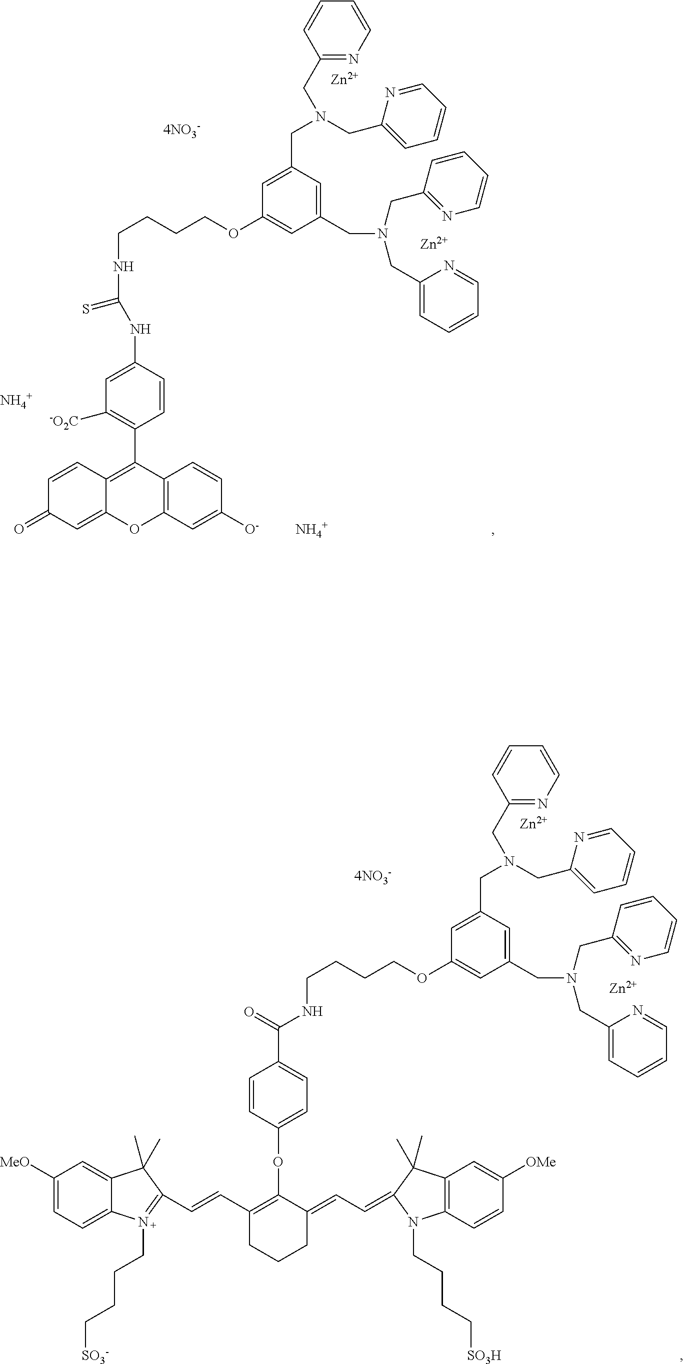

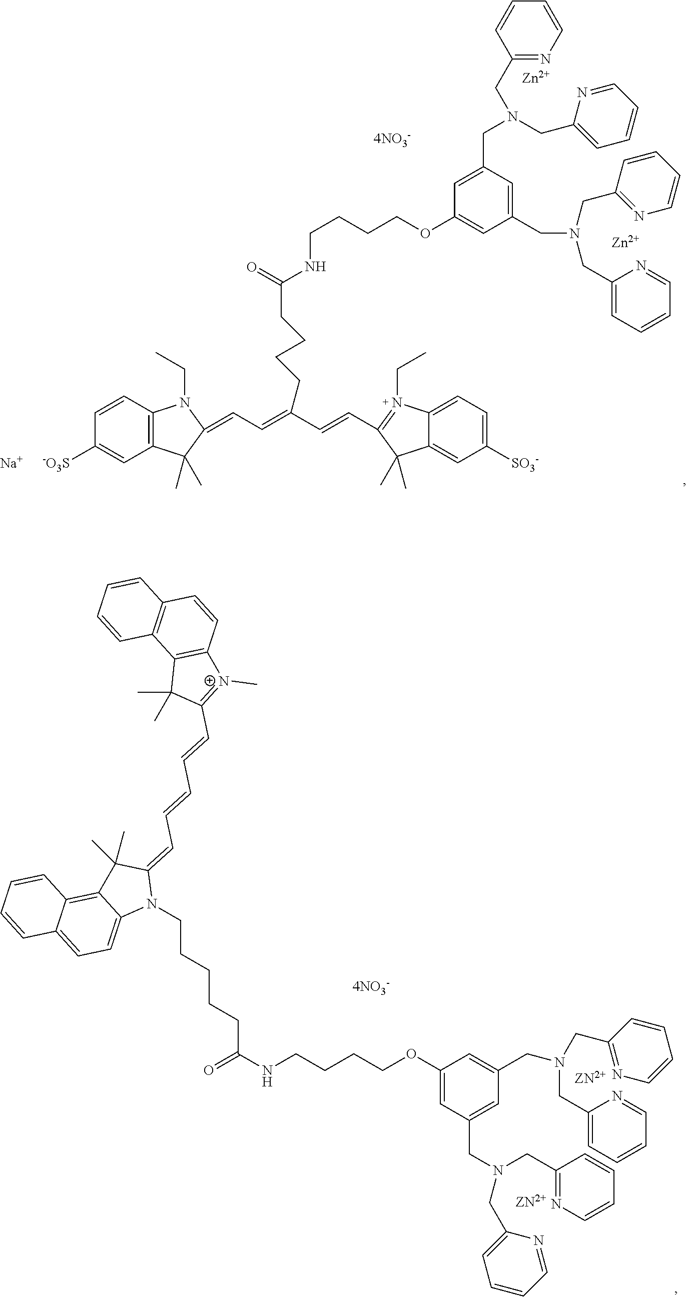

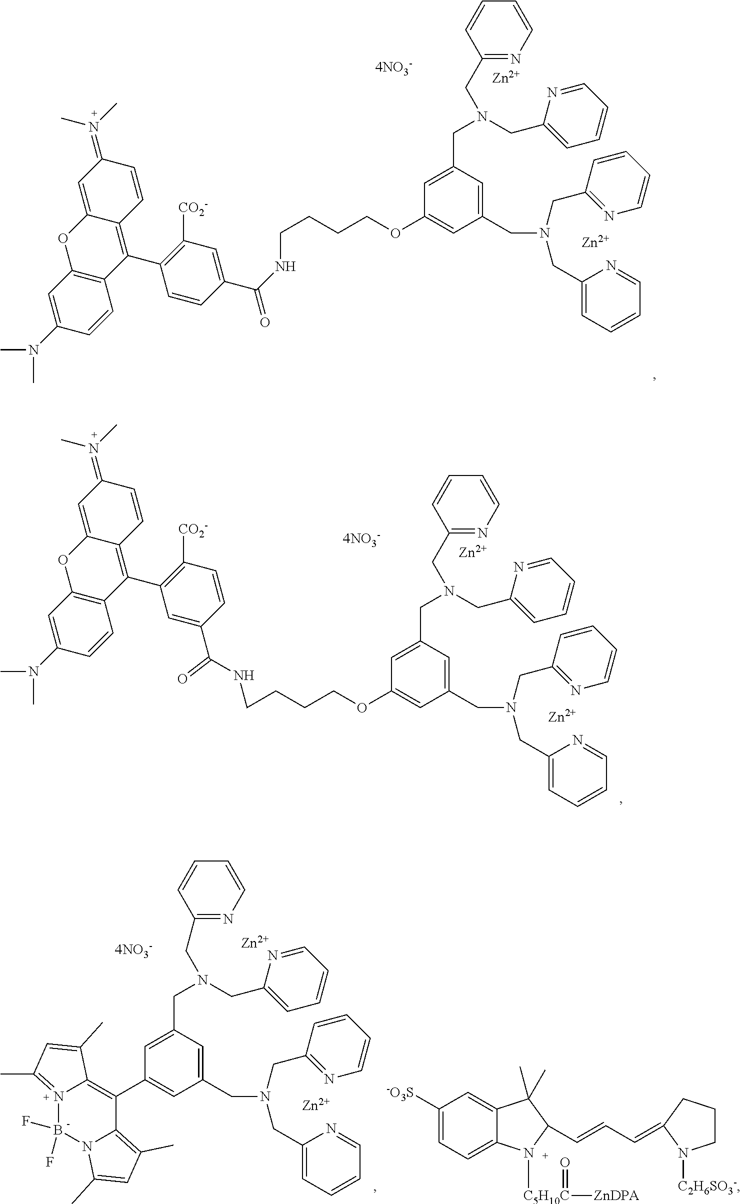

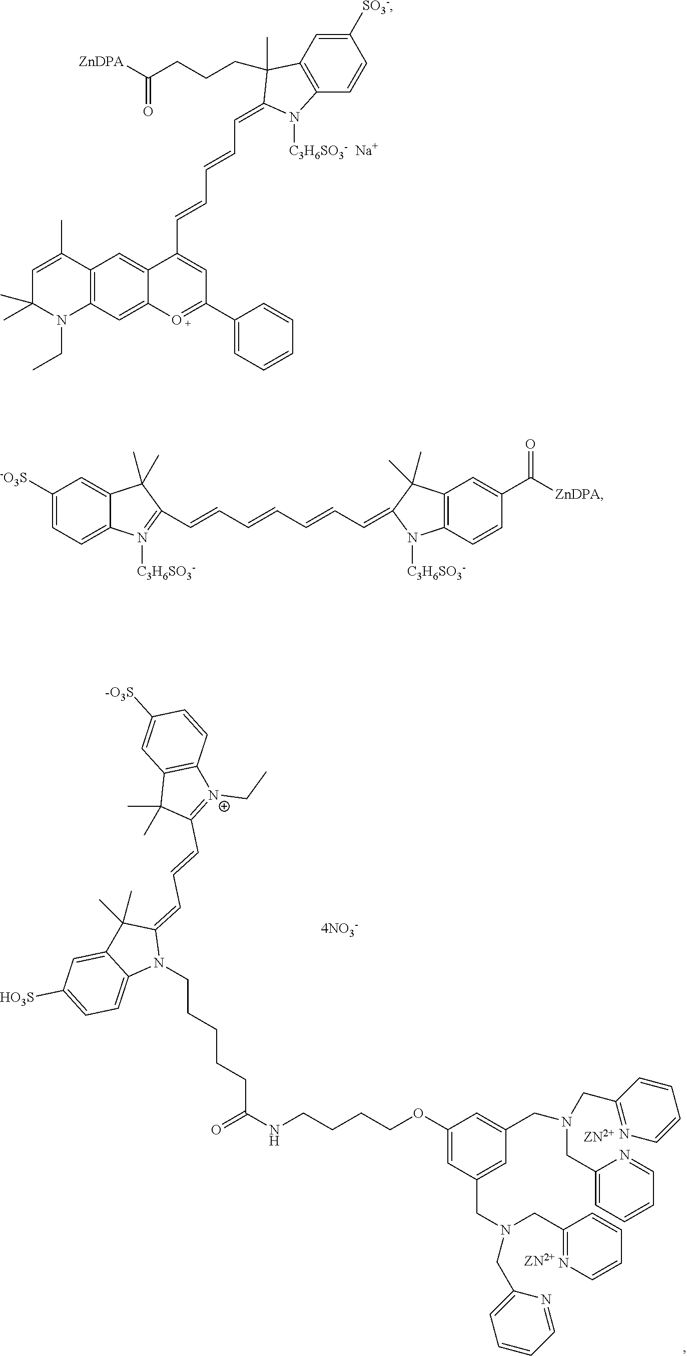

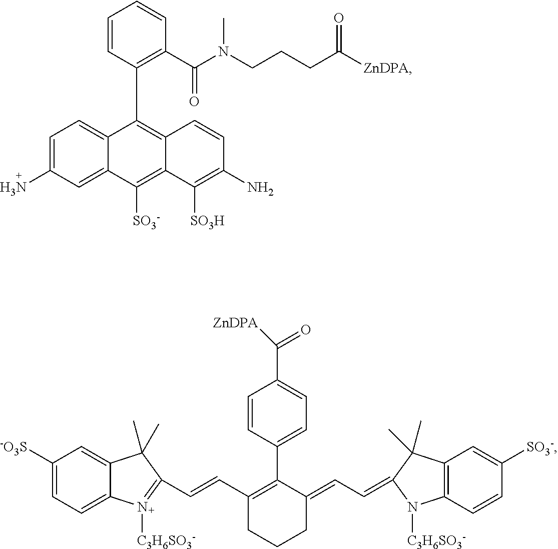

or a combination thereof,
where ZnDPA has the following structure:

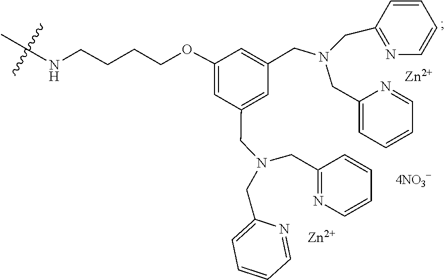 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

dilating the pupil of the eye of the individual; and imaging the eye of the individual. The method of the present disclosure involves administration via topical application to the eye (e.g., via application of one or more eye drops). Methods of the present disclosure do not involve administration via intraocular injection.

Methods of the present disclosure may be performed in vivo on a subject in need of treatment having or suspected of having an eye disease and/or eye injury.

For detecting fluorescence groups of the compounds of the present disclosure, various methods known in the art may be used. For example, optical molecular imaging technologies use light emitted through fluorescence and the fluorescence group of interest can be excited by particular wavelength resulting in the emission of light that can be visualized and recorded by camera such as charge-coupled device camera. This technique can be incorporated into available diagnostic instruments including biomicroscopy (slit lamp), optical coherence tomography, confocal laser scanning microscopy, adaptive optics scanning laserophthalmoscopy, ophthalmoscopy and fundus camera. Imaging may be performed immediately after administration, 1 minute after administration, 5 minutes after administration, 10 minutes after administration, 30 minutes after administration, 1 hour after administration, 2 hours after administration, 3 hours after administration, 4 hours after administration, 5 hours after administration, 6 hours after administration, 7 hours after administration, 8 hours after administration, 9 hours after administration, 10 hours after administration, or up to 50 hours after administration. In various examples, the eye drops are applied and imaging occurs within 24 hours. In various examples, imaging occurs 3 minutes to 24 hours following eye drop administration of a composition of the present disclosure. In various examples, after or around 72 hours, compounds are cleared from the area.

In various examples, the fluorescent group has an emission max range of 440-900 nm and an absorbance max range of 380-880 nm. The fluorescence emission may be quantified to determine the amount of retinal apoptosis.

Imaging may be performed with, for example, a Micron-IV retinal camera or a similar camera. Prior to imaging, eyes may be covered with artificial tears. Images may be acquired at different focal planes starting from the cornea and moving to the back of the eye. Fluorescence intensity may be quantified using methods known in the art (e.g., ROI using ImageJ).

The dose of the composition comprising a compound of the present disclosure and a pharmaceutical agent may necessarily be dependent upon the needs of the individual to whom the composition of the disclosure is to be administered. These factors include, for example, the weight, age, sex, medical history, and nature and stage of the disease for which a therapeutic or prophylactic effect is desired. The compositions may be used in conjunction with any other conventional treatment modality designed to improve the disorder for which a desired therapeutic or prophylactic effect is intended.

Compounds and compositions comprising compounds may be dosed at various dosages. The compositions may have various suitable concentrations. Examples of the concentration include, but are not limited to, 0.1 mM to 1.0 M, including every 0.01 mM value and range therebetween (e.g., 0.10 mM to 1.0 M, 0.1 mM to 10 mM, 1 mM to 10 mM, 0.1-15 mM, 0.1 mM to 50 mM). In various examples, a composition may be administered as an eye drop at a concentration 0.5-1 mM, wherein the individual is administered 1-10 drops (1, 2, 3, 4, 5, 6, 7, 8, 9, or 10 drops).

A method of the present disclosure may comprise additional imaging to determine if an individual is recovering from and/or after a therapeutic treatment.

For example, a method of determining effectiveness of a treatment comprises i) administering an eye drop of claim 20 to an eye of an individual, ii) dilating the eye of the individual, iii) imaging the eye of the individual, iv) waiting a period of time, v) optionally repeating steps i) and ii) or ii), vi) imaging the eye of the individual, and vii) comparing the images obtained from imaging, where the comparison is used to the effectiveness of treatment. Steps i) and ii) may be repeated when additional compound or compounds of the present disclosure is/are needed for imaging or just step ii) may be repeated if there is still suitable detectable compound or compounds of the present disclosure and additional compound or compounds would not be needed for imaging, however dilation would be required for imaging.

In an aspect, the present compounds and compositions may be used as research tools.

In various examples, the compounds and/or compositions of the present disclosure are used to determine if the compounds of the present disclosure or other compounds induce or cause injury to any part of the retina and/or induce apoptosis of retinal cells.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like. The compounds or compositions of the present disclosure may be administered to individuals for example, in pharmaceutically-acceptable carriers, which facilitate transporting the compounds from one organ or portion of the body to another organ or portion of the body.

The steps of the method described in the various examples disclosed herein are sufficient to carry out the methods of the present disclosure. Thus, in an example, the method consists essentially of a combination of the steps of the methods disclosed herein. In another example, the method consists of such steps.

The following Statements provide examples of compounds, compositions, and methods of the present disclosure:

Statement 1. A compound having the following structure:

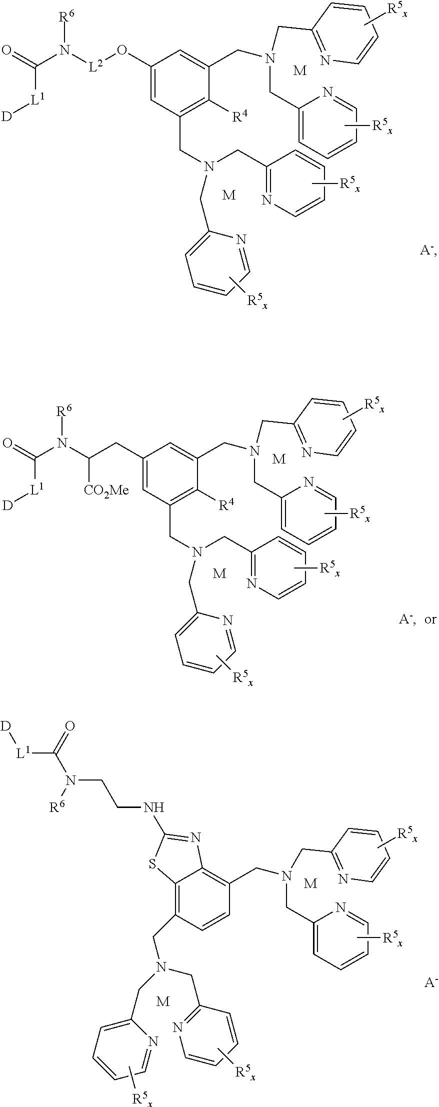

where D is chosen from:

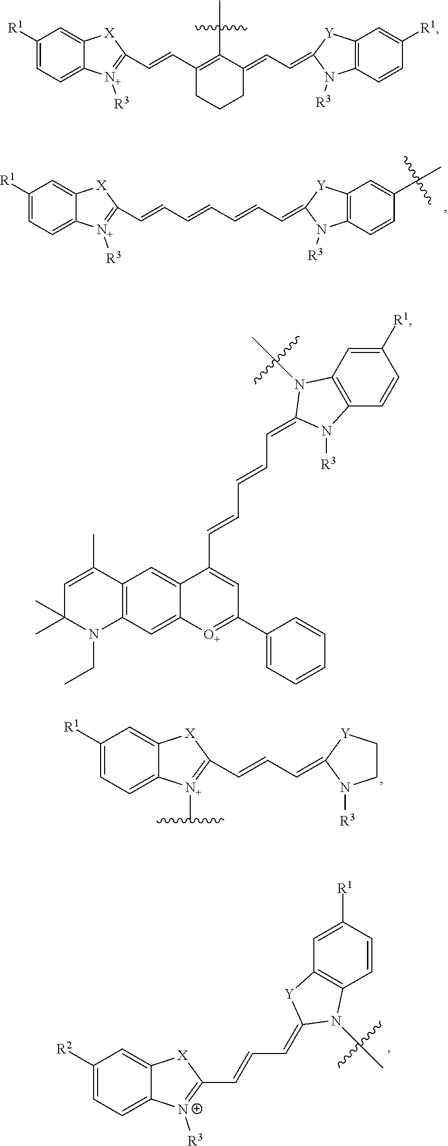

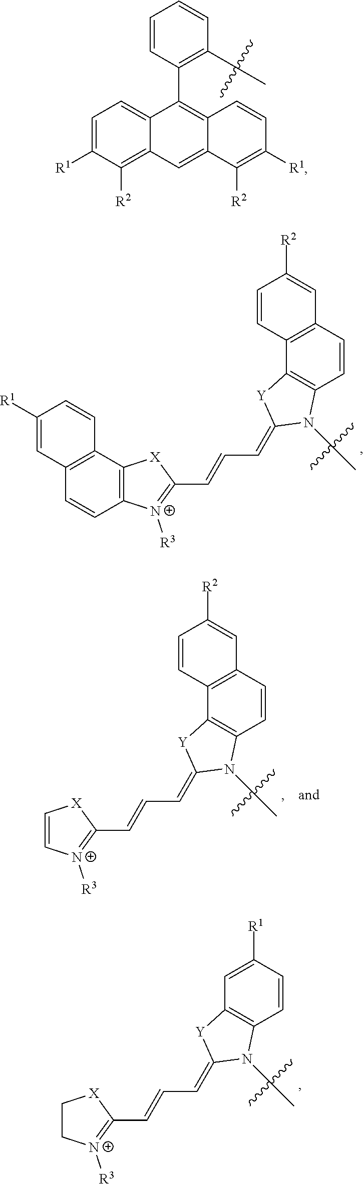

R¹ and R² are polar groups that may be the same or different; R³ is chosen from alkyl groups (e.g., methyl, ethyl, propyl, isopropyl, butyl, pentyl, hexyl, and the like), polyethylene glycol groups, and C₁ to C₁₂ alkyl sulfonic acid groups (e.g., methyl sulfonic acid groups, ethyl sulfonic acid groups, propyl sulfonic acid groups, butyl sulfonic acid groups, pentyl sulfonic acid groups, and the like); R⁴ is chosen from —H and —OH; R⁵ is chosen from —H, alkyl groups (e.g., methyl, ethyl, propyl, isopropyl, butyl, pentyl, hexyl, and the like), amine groups, amide groups, carbamide groups, carbamate groups, and halogen groups; R⁶ is chosen from —H and alkyl groups (e.g., methyl, ethyl, propyl, isopropyl, butyl, pentyl, hexyl, and the like); L¹ and L² are independently optional linking groups that may be the same or different; A is one or more counterions; M is a divalent cation; x is individually at each occurrence is 1-4; Z is C(R₆); and X and Y are independently C(R⁶)₂, O or S, where each R⁶ is the same or different.
Statement 2. The compound according to Statement 1, where the polar groups are individually chosen from protonated sulfonic acid groups, deprotonated sulfonic acid groups (e.g., sulfonic acid salts, such as, for example, sodium or potassium salts), C₁ to C₁₂ alkyl sulfonic acid groups, polyethylene glycol groups, sugar groups, quaternary ammonium groups, phosphonic acid groups, and combinations thereof. When polar groups are ionized, they may have various counterions (e.g., sodium ions, potassium ions, and the like). Ionized compounds may be pharmaceutically acceptable salts.
Statement 3. The compound according to Statements 1 or 2, where linking groups are chosen from ethyl groups, propyl groups, butyl groups, pentyl groups, hexyl groups, PEG having to 2-6 repeat units, and the like, and combinations thereof.
Statement 4. The compound according to any one of the preceding Statements, where the divalent cations are chosen from Mn²⁺, Fe²⁺, Co²⁺, Ni²⁺, Cu²⁺, Zn²⁺, and combinations thereof.
Statement 5. The compound according to any one of the preceding Statements, where the compound has the following structure:

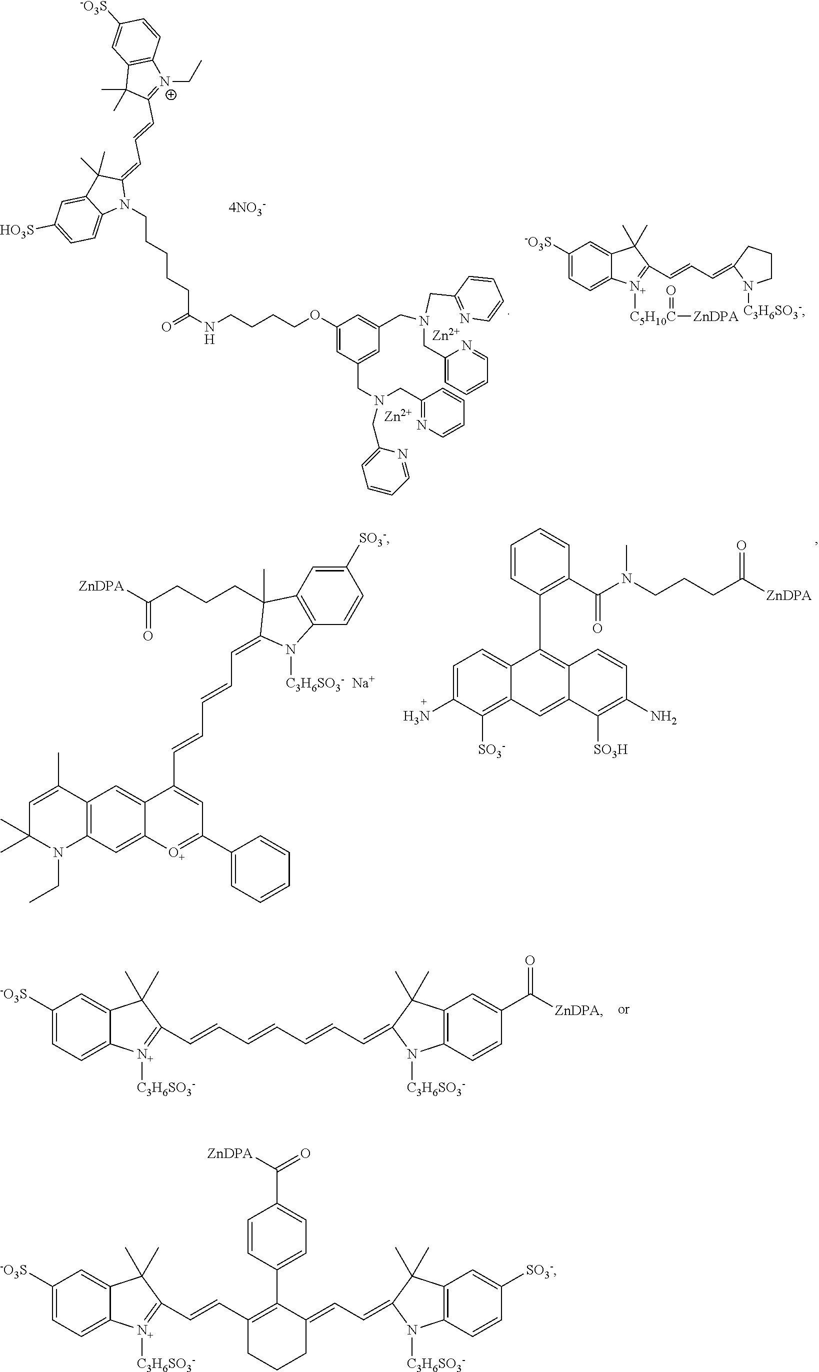

where ZnDPA has the following structure:

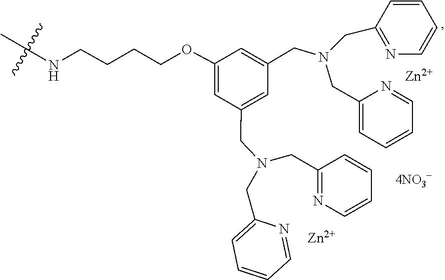 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

where the alkyl sulfonic acid groups (e.g., CₓH_{y}SO₃⁻ and CₓH_{y}SO₄) may be linear alkyl groups (e.g., n-propyl, n-pentyl).
Statement 6. A composition comprising a compound according to any one of the preceding Statements and a pharmaceutically acceptable carrier. The composition may be an aqueous solution comprising ≤20% DMSO v/v, ≤19% DMSO v/v, ≤18% DMSO v/v, ≤17% DMSO v/v, ≤16% DMSO v/v, ≤15% DMSO v/v, ≤14% DMSO v/v, ≤13% DMSO v/v, ≤12% DMSO v/v, ≤11% DMSO v/v, ≤10% DMSO v/v, ≤9% DMSO v/v, ≤8% DMSO v/v, ≤7% DMSO v/v, ≤6% DMSO v/v, ≤5% DMSO v/v, ≤4% DMSO v/v, ≤3% DMSO v/v, ≤2% DMSO v/v, ≤1% DMSO v/v, ≤0.9% DMSO v/v, ≤0.8% DMSO v/v, ≤0.8% DMSO v/v, ≤0.7% DMSO v/v, ≤0.6% DMSO v/v, ≤0.5% DMSO v/v, ≤0.4% DMSO v/v, ≤0.3% DMSO v/v, ≤0.2% DMSO v/v, ≤0.1% DMSO v/v, or no DMSO.
Statement 7. The composition according to Statement 6, where the compound has the following structure:

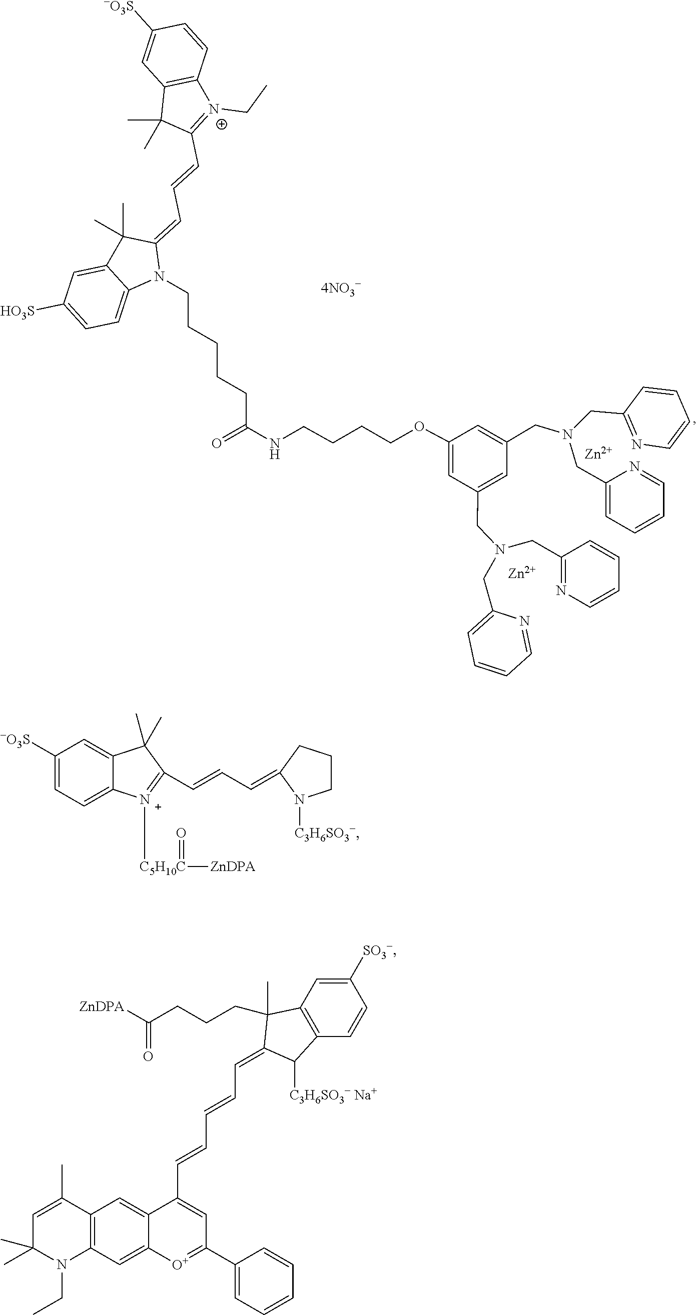

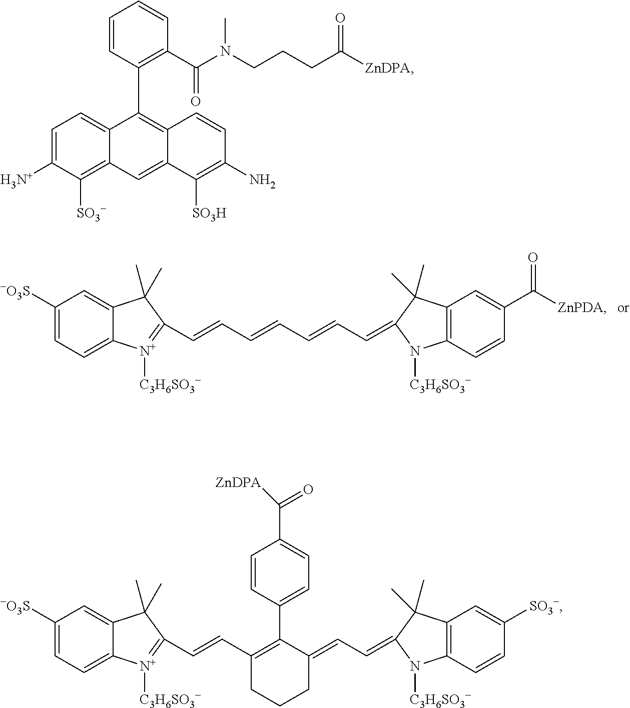

where ZnDPA has the following structure:

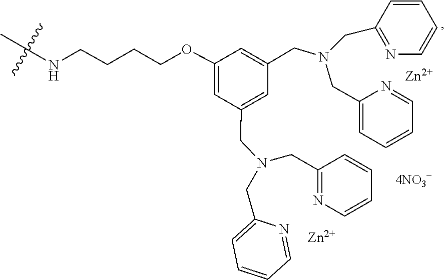 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

where the alkyl sulfonic acid groups (e.g., CₓH_{y}SO₃⁻ and CₓH_{y}SO₄) may be linear alkyl groups (e.g., n-propyl, n-pentyl).
Statement 8. A method of determining the presence of and/or amount of retinal apoptosis in an individual in need of treatment, comprising: i) administering to an eye of the individual an effective amount of a first composition according to any one of Statements 6 or 7; a second composition comprising a pharmaceutically acceptable carrier and a compound having the following structure:

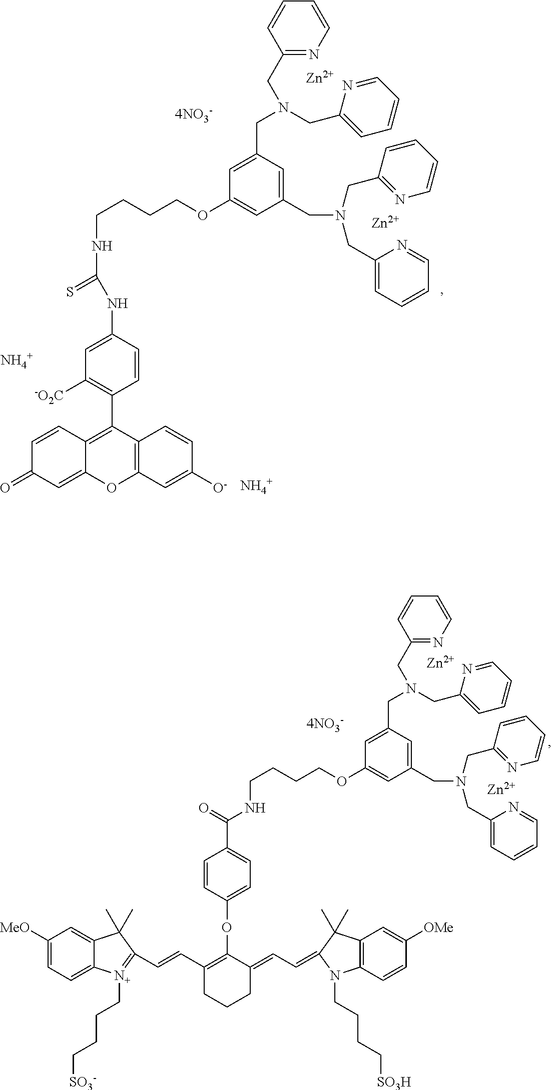

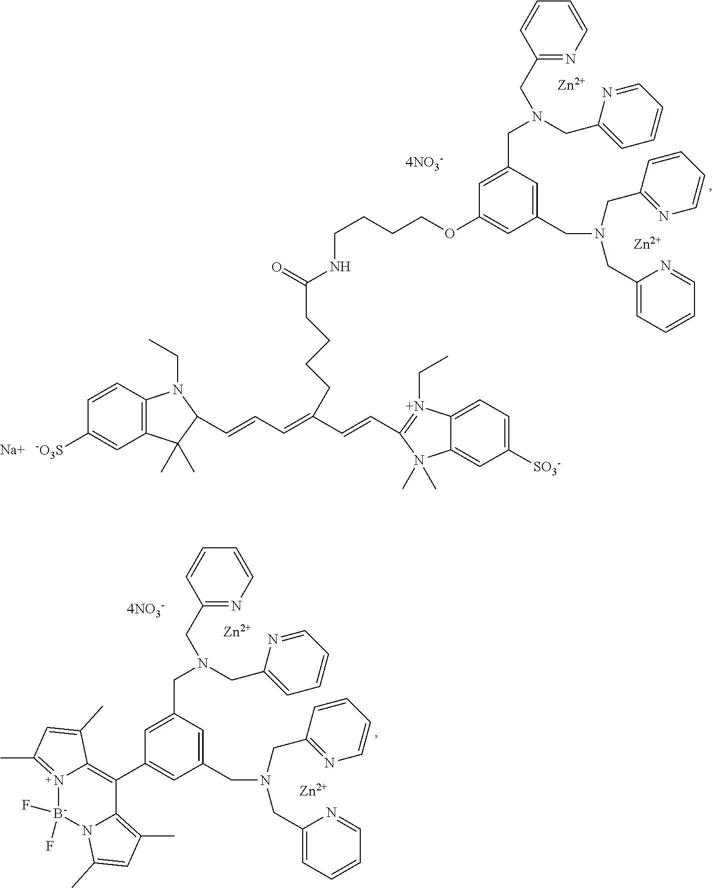

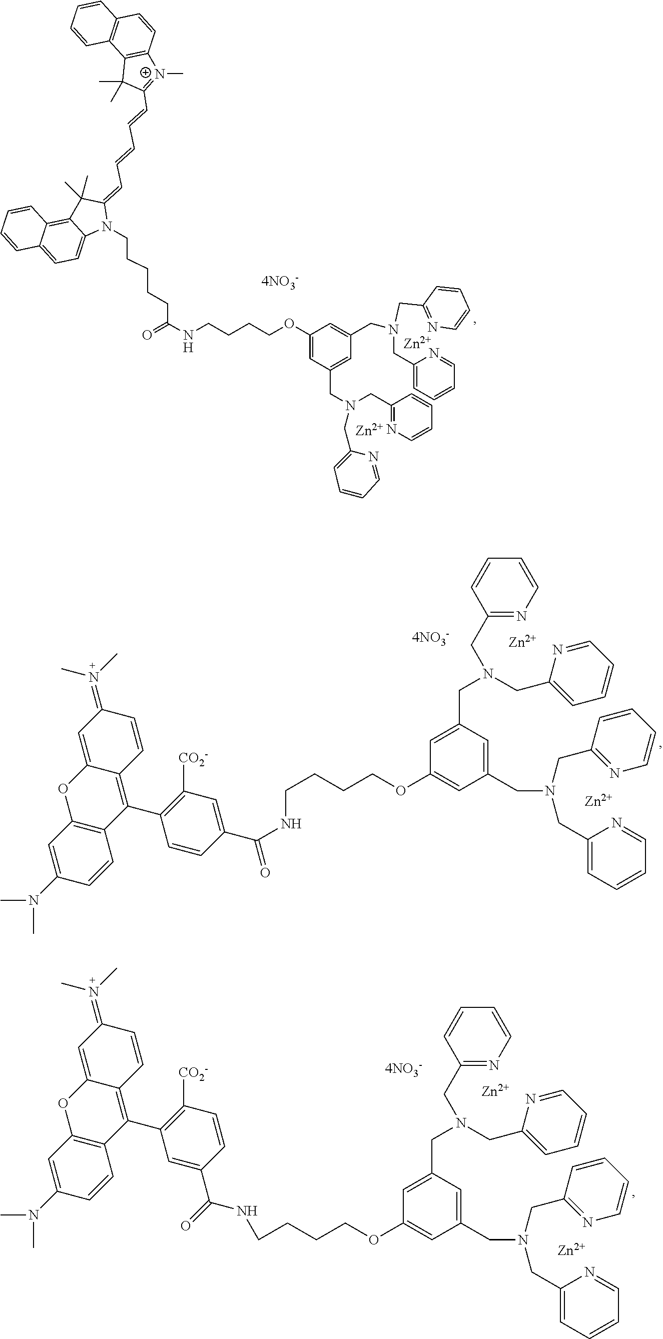

or a combination thereof, or a combination of the first composition and the second composition; ii) dilating the pupil of the eye of the individual; and iii) imaging the eye of the individual, where the administration is not via intraocular injection. The composition may comprise having ≤20% DMSO, ≤19% DMSO, ≤18% DMSO, ≤17% DMSO, K 16% DMSO, ≤15% DMSO, ≤14% DMSO, ≤13% DMSO, ≤12% DMSO, ≤11% DMSO, ≤10% DMSO, ≤9% DMSO, ≤8% DMSO, ≤7% DMSO, ≤6% DMSO, ≤5% DMSO, ≤4% DMSO, ≤3% DMSO, ≤2% DMSO, ≤1% DMSO, ≤0.5% DMSO, ≤0.1% DMSO, or no DMSO. The composition may be an aqueous solution (e.g., saline or a buffered solution). The method may be performed in vivo.
Statement 9. The method according to Statement 8, where the first composition and/or second composition are administered as an eye drop.
Statement 10. The method according to Statements 8 or 9, where the compound(s) of the first composition and/or second composition have a concentration of 0.1-15 mM, including all 0.01 mM values and ranges therebetween.
Statement 11. The method according to any one of Statements 8-9, where the imaging comprises imaging via a retinal imaging system (e.g., a Micron-IV retinal camera or a similar device).
Statement 12. The method according to any one of Statements 8-11, where the imaging comprises excitation with electromagnetic radiation (e.g., exposing the individual in need of treatment with electromagnetic radiation).
Statement 13. The method according to Statement 12, where fluorescence emission is observed and occurs in the range of 440-900 nm, including all nm integer values and ranges therebetween.
Statement 14. The method according to any one of Statements 13, where the fluorescence emission is quantified.
Statement 15. The method according to any one of Statements 8-14, where the imaging is performed 1 minute after administration, 5 minutes after administration, 10 minutes after administration, 30 minutes after administration, 1 hour after administration, 2 hours after administration, 3 hours after administration, 4 hours after administration, 5 hours after administration, 6 hours after administration, 7 hours after administration, 8 hours after administration, 9 hours after administration, 10 hours after administration, or up to 50 hours after administration. In various examples, the eye drops are applied and imaging occurs within 24 hours. In various examples, imaging occurs 3 minutes to 24 hours following eye drop administration of a composition of Statements 6 or 7.
Statement 16. The method according to any one of Statements 8-15, where the individual in need of treatment has primary open angle glaucoma, acute closed angle glaucoma, chronic closed angle glaucoma, myopic retinopathies, macular edema, genetic disease of the retina and macular, pars planitis, Posner Schlossman syndrome, Bechet's disease, Vogt-Koyanagi-Harada syndrome, hypersensitivity reactions, toxoplasmosis chorioretinitis, inflammatory pseudo-tumor of the orbit, chemosis, conjunctival venous congestion, periorbital cellulitis, acute dacryocystitis, nonspecific vasculitis, sarcoidosis, cytomegalovirus infection, diabetic retinopathy, age-related macular degeneration, or the like, or a combination thereof.
Statement 17. The method according to any one of Statements 8-16, where the first composition and/or second composition are not detectable after 72 hours.
Statement 18. The method according to any one of Statements 8-17, where the composition comprises:

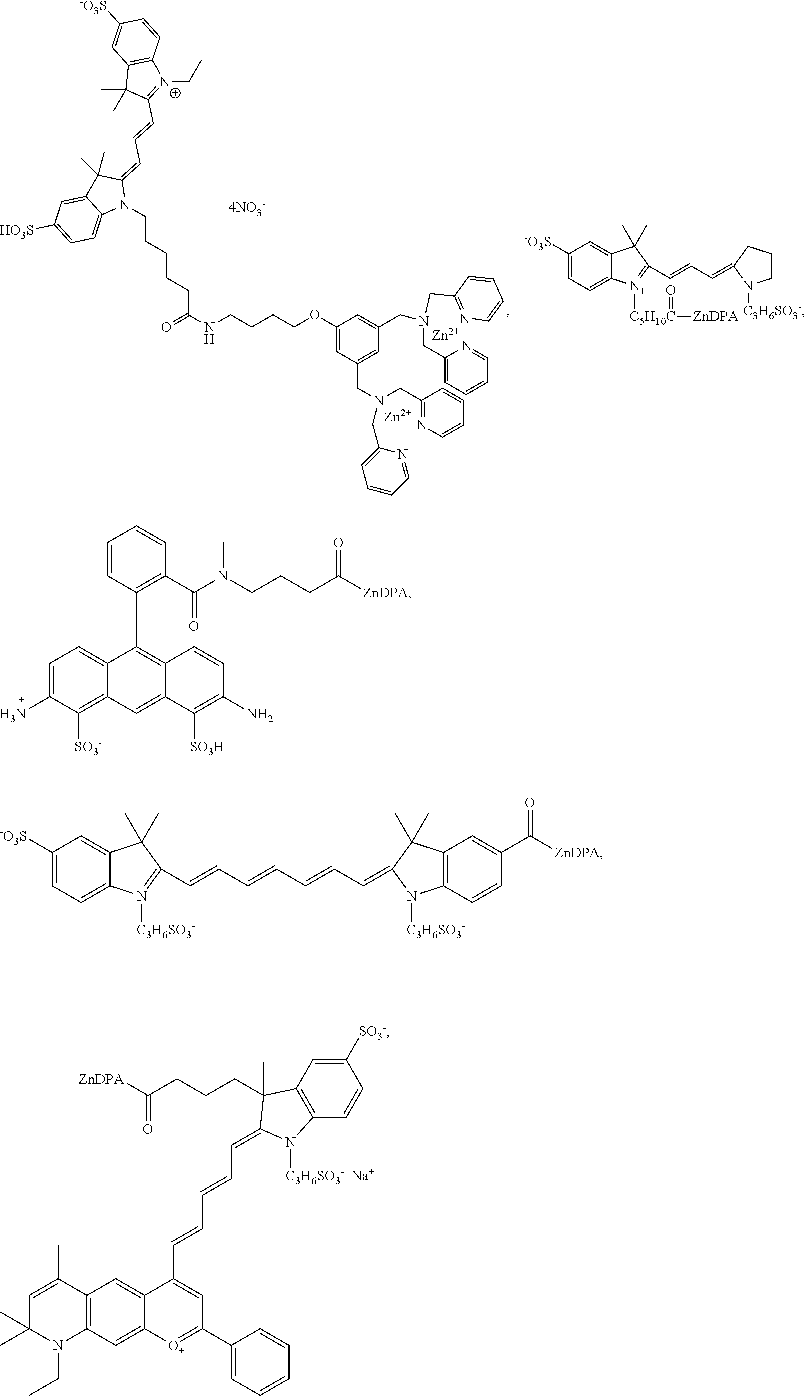

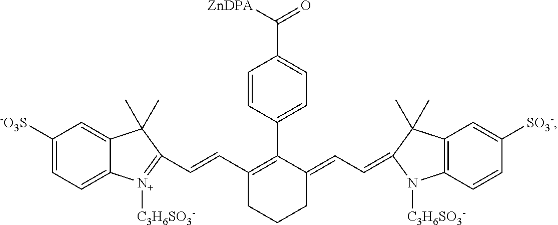

or a combination thereof,
where ZnDPA has the following structure:

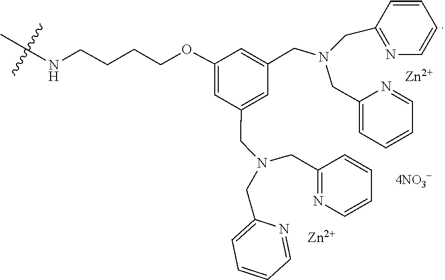 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

Statement 19. The method according to any one of Statements 8-18, where the composition comprises:

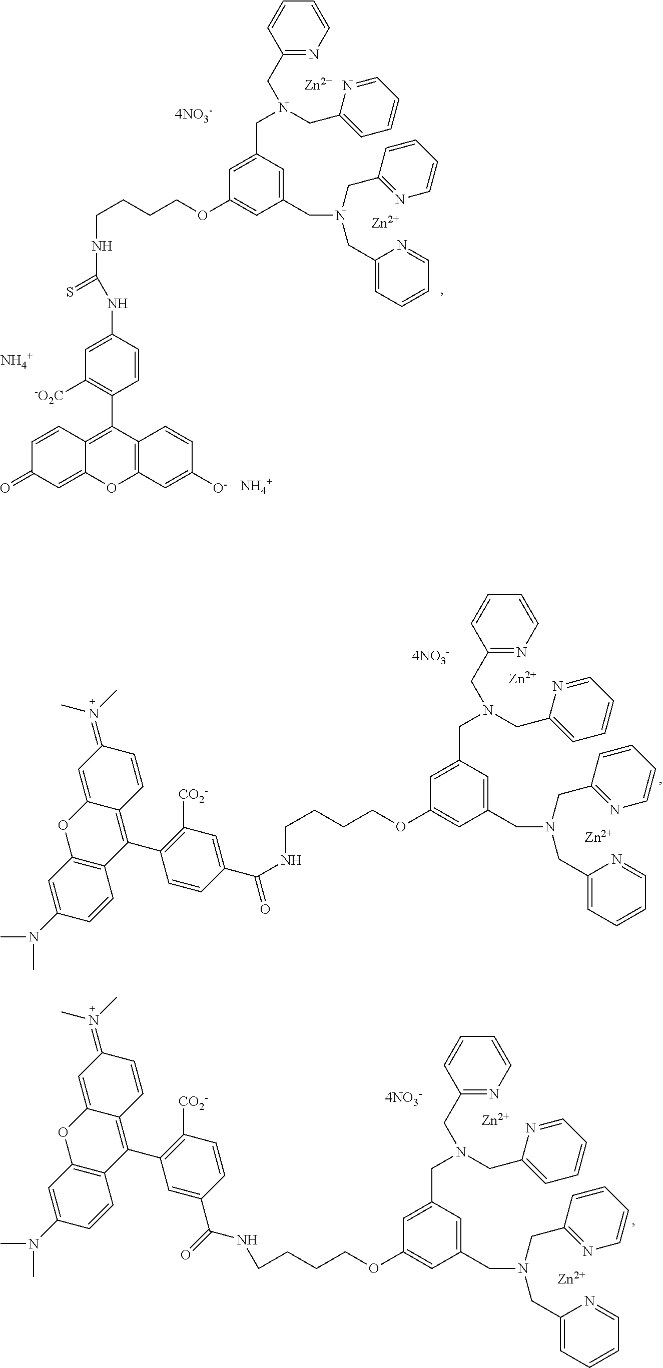

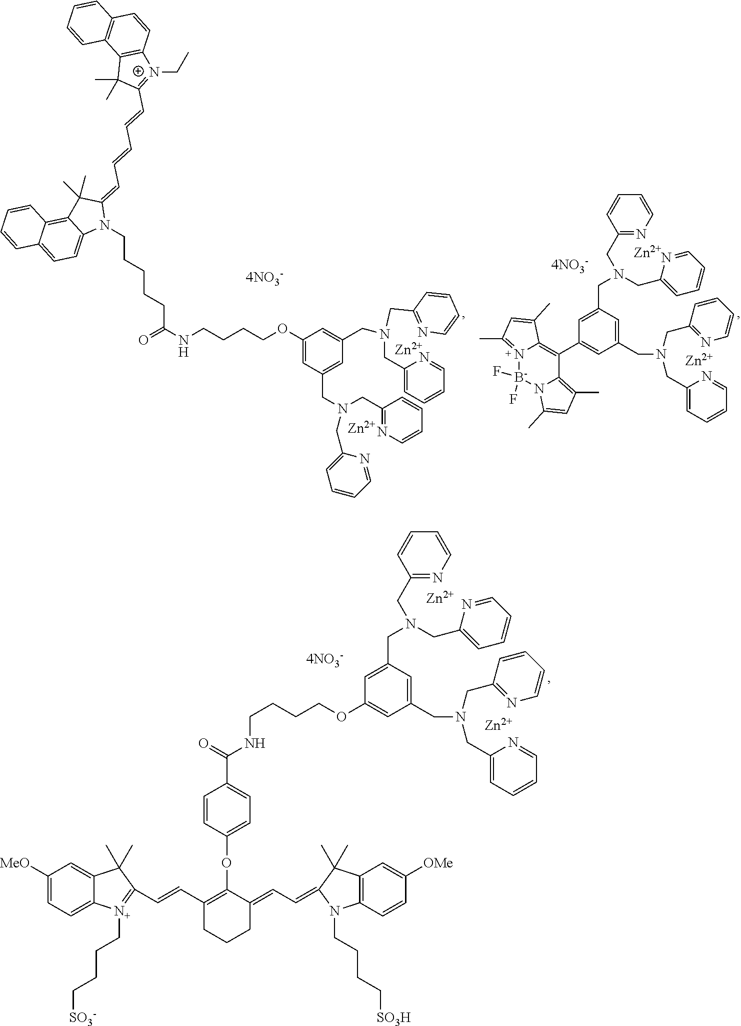

or a combination thereof.
Statement 20. An eye drop comprising a compound according to any one of Statements 1-7. The eye drop may be a composition of Statements 6 or 7.
Statement 21. The eye drop according to Statement 20, where the compound is chosen from:

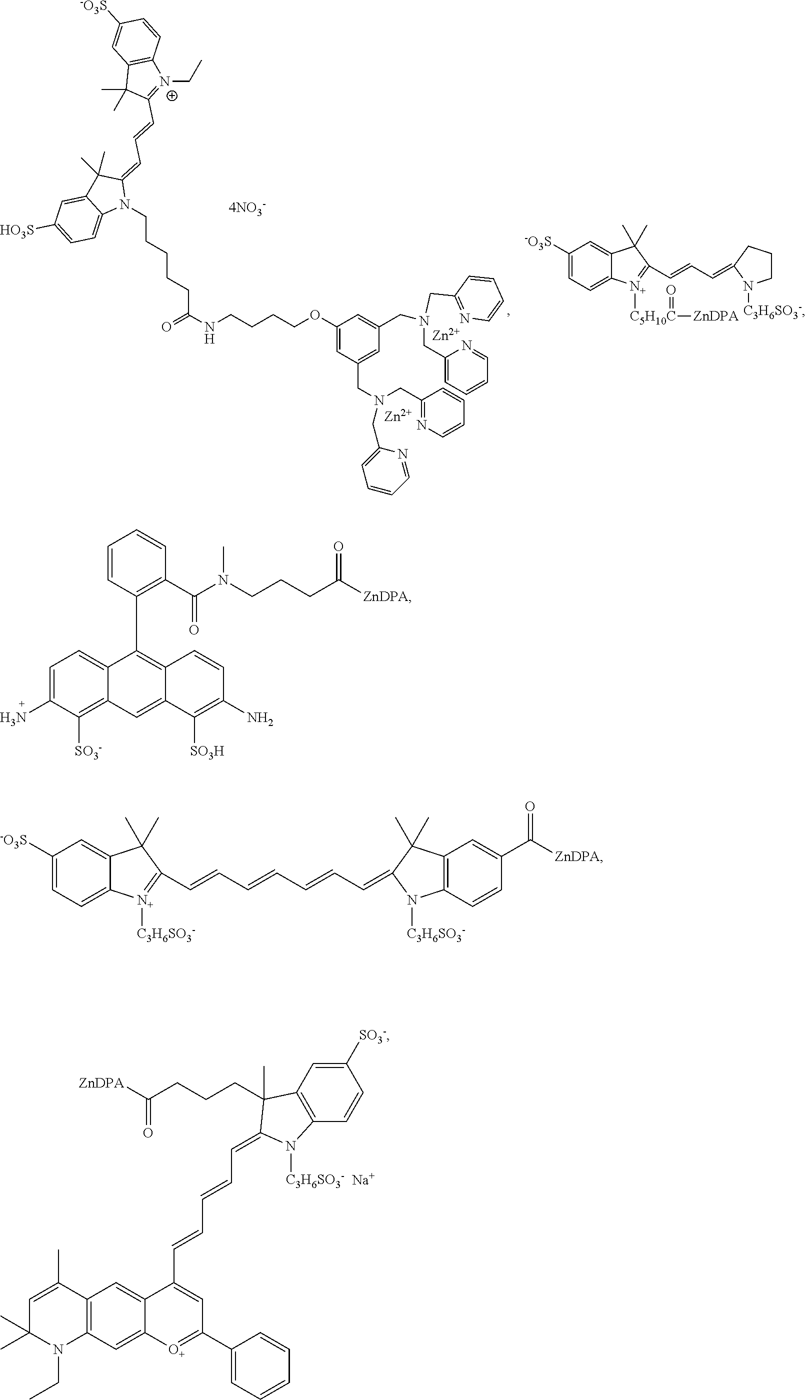

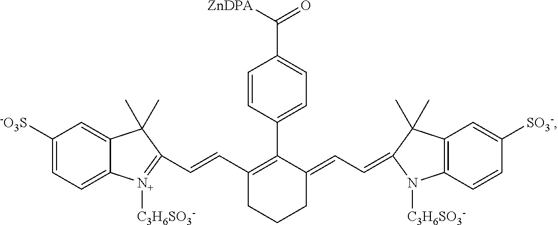

and combinations thereof.
where ZnDPA has the following structure:

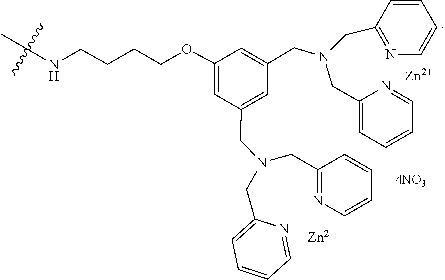 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

Statement 22. A method of determining effectiveness of a treatment for eye injury comprising: i) administering an eye drop of claim 20 to an eye of an individual, ii) dilating the eye of the individual, iii) imaging the eye of the individual, iv) waiting a period of time, v) optionally repeating steps i) and ii) or ii), vi) imaging the eye of the individual, and vii) comparing the images obtained from imaging, where the comparison is used to the effectiveness of the treatment.

The following examples are presented to illustrate the present disclosure. They are not intended to be limiting in any way.

### Example 1

This example provides a description of the synthesis of compounds of the present disclosure.

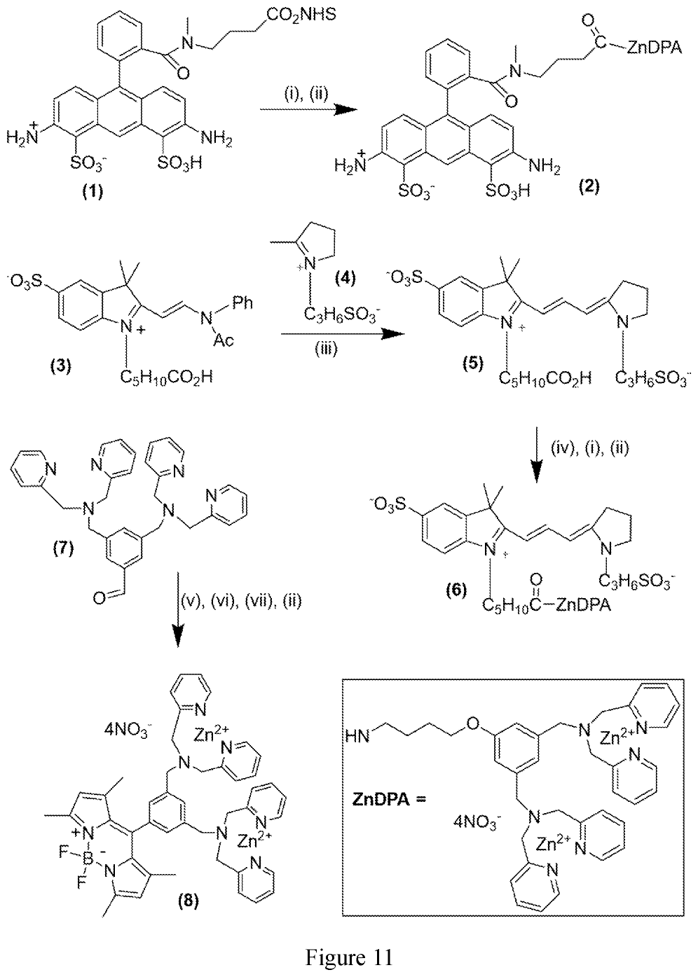

Synthesis of water soluble 488 nm excitation dyes: Three water soluble compounds will be synthesized as shown in FIG. 11. Compound (2) will be prepared by reacting the hydrophilic dye (1) (fluorescence ex/em=500/520 nm and excellent water solubility) in its activated N-hydroxysuccinimide (NHS) ester form with DPA-amine (prepared in 5 synthetic steps from dimethyl-5-hydroxy-isophthalate) at room temperature overnight. The resulting conjugate will be purified by C18 reverse phase silica gel column chromatography or semi-preparative reverse phase HPLC and then treated with 2 mole equivalents of zinc nitrate dihydrate in ethanol for 60 minutes and then the solvent removed to provide (2).

Compound (6), also containing two water solubilizing sulfonic acid groups, is expected to have fluorescence ex/em properties of 480/495 nm. To provide (6), (3) will be synthesized by alkylating 5-sulfo-2-3,3-trimethylindolenine with 6-bromohexanoic acid and then treating the resulting quaternary ammonium salt with N,N-diphenylformamide in acetic anhydride. Heating (3) with the quaternary ammonium salt (4) in the presence of triethylamine in dichloromethane will provide dye (5). (5) will be purified by reverse phase silica gel chromatography using methanol/water solvent systems. Next, (5) will be converted to its activated NHS ester form in situ by reaction with N,N-dicyclohexylcarbodiimide (DCC) and NHS in DMF and treated with DPA-amine to provide apo-(6) which will be purified by reverse phase chromatography. Incorporation of zinc to form the dye (6) will be achieved as indicated above for dye (2).

Compound (8) with fluorescence ex/em=499/520 nm is a previously reported optical probe that combines a zinc (II)-dipicolylamine targeting unit and a BODIPY chromophore and has been used for fluorescence imaging of bacteria. Without intending to be bound by any particular theory, it is expected that this dye will be highly water soluble and non-phototoxic with a high fluorescence quantum yield (#=0.53). It will be prepared in 5 steps in ˜30% overall yield according to the methods described herein.

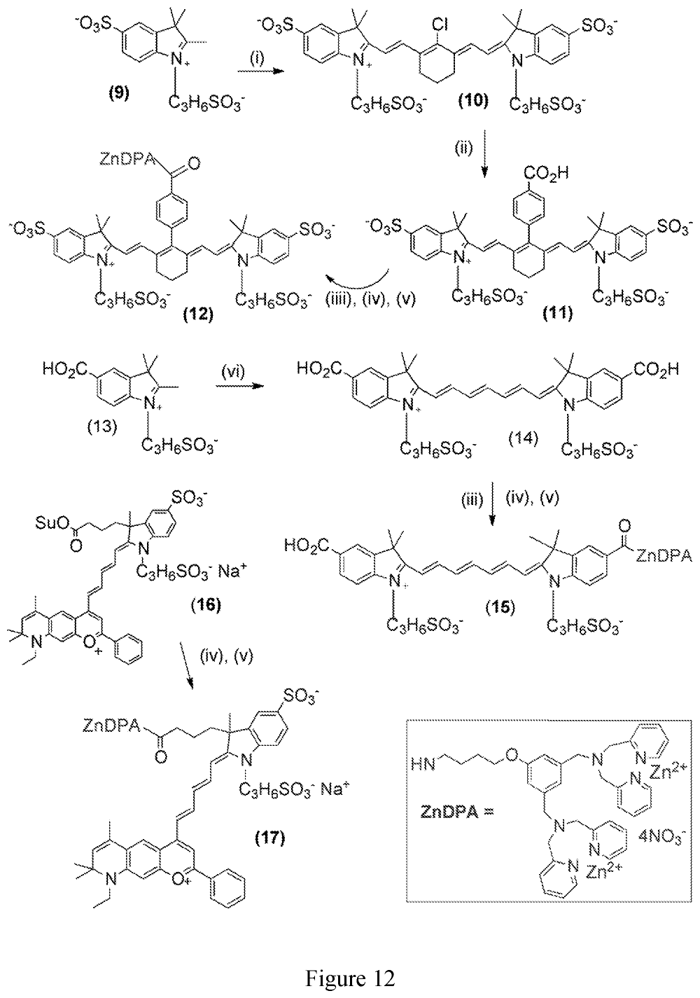

Synthesis of water soluble 780 nm excitation dyes: Three candidate dyes will be synthesized as shown in FIG. 12. Compound (12) (expected fluorescence ex/em=784/801 nm) is designed to be highly water soluble, having 4 sulfonic acid groups. To prepare (12), first compound (9) is prepared by heating 5-sulfo-2,3,3-trimethylindoleine with 1,3-propane sultone. Coupling of 2 mole equivalents of (9) with the commercially available bridging unit, N-[(3-(anilinomethylene)-2-chloro-1-cyclohexen-1-yl) methylene] aniline monohydrochloride in the presence of sodium acetate will produce (10) which will be purified by reverse phase column chromatography. Palladium-catalyzed cross coupling of (10) with 4-carboxyphenylboronic acid (Suzuki-Miyaura reaction) will provide compound (11). Elaboration of (11) will then proceed via conversion to its activated ester, reaction with DPA-amine, and complexation with zinc to provide (12).

To prepare (15), compound (13) will be prepared by heating 5-carboxy-2,3,3-triemethylindolenine with propane sultone. Then, 2 molar equivalents of (13) will be reacted with N-[5-(phenylamino)-2,4-pentadienylidene] aniline monochloride (Millipore-Sigma, Milwaukee, Mich.) by heating in acetic anhydride at 130° C. to provide the dicarboxylic acid dye (14) which will be purified by chromatography. Elaboration of (14) to (15) will proceed using the same reactions described for conversion of (11) to (12).

Dye (16) (fluorescence ex/em=771/793 nm) was studied in a Phase 1 clinical trial for the real-time detection of neuronal cell death in patients with glaucoma. When administered by i.v. injection it was found to be safe. Dye (16) in its activated ester form will be reacted with DPA-amine in DMF and the product purified by chromatography and treated with 2 equivalents of zinc nitrate as before to provide candidate (17).

Characterization will be performed as described in Table 1.

TABLE 1
Methods of characterization of fluorescent dyes and chemical
intermediates.

| Data Sought | Methodology |
|---|---|
| Spectral | Absorbance spectra, extinction coefficients (ε) determined by Beer’s law |
| Properties | at low concentrations (0.1-0.6 μM) & fluorescence excitation/emission |
| | spectra will be obtained. |
| Solubility | Solubility in water will be determined by visually inspecting the |
| | solutions for particulates. |
| Quantum Yield | To be calculated as described using ICG, which has the value of 0.078 in |
| (ϕ) | in methanol and fluorescein, which has the value of 0.92 in aqueous |
| | solution. |
| Purity | To be determined by reverse phase HPLC using a methanol-water and |
| | 0.1% TFA gradient system. |
| NMR | Samples will be tested by proton NMR. |
| Mass Spec | Samples for MS will be sent for testing to AA Labs, Inc. |
| Lipophilicity | The octanol-water partition constants will be determined using the |
| (Log P) | shake-flask method as described. |

### Example 2

This example provides a description of the synthesis of compounds of the present disclosure.

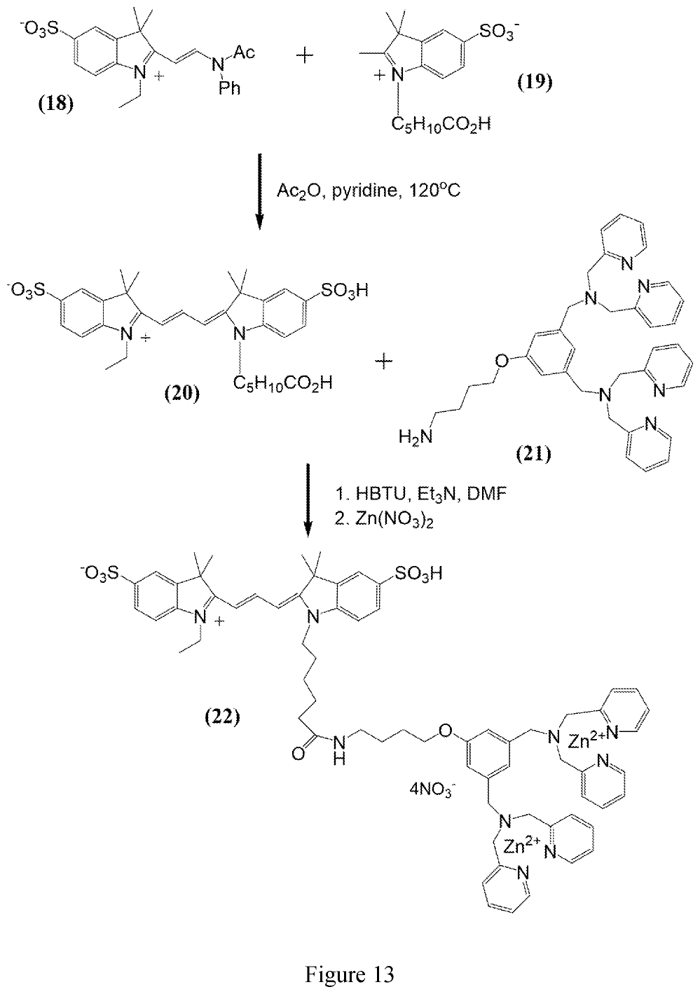

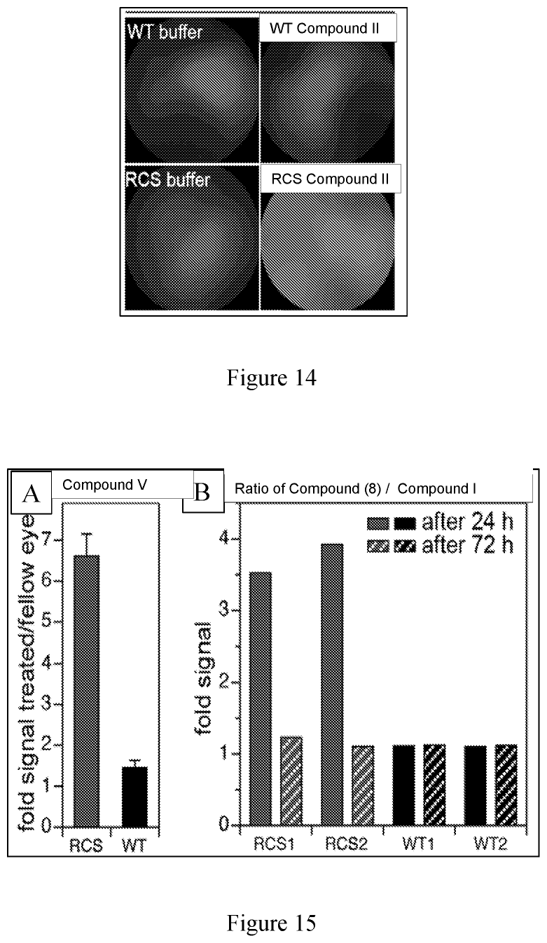

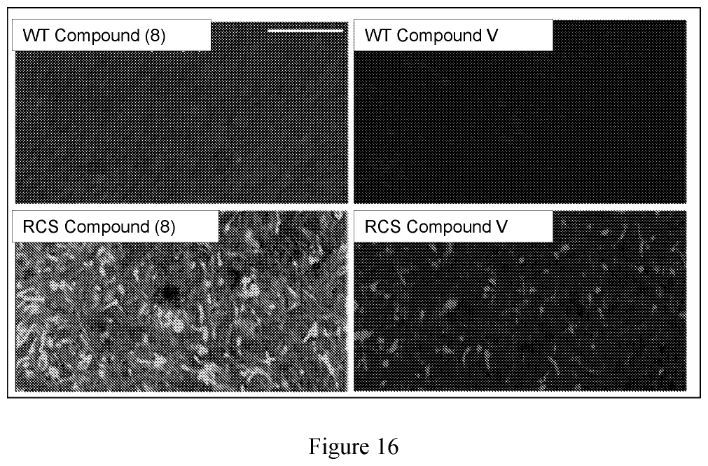

The following example refers to the synthetic scheme presented in FIG. 13

Preparation of Compound (18). 2,3,3-trimethyl-3H-indole-5-sulfonic acid was prepared and alkylated with ethyl iodide to afford 1-ethyl-2,3,3-trimethyl-3H-indolinium-5-sulfonate according to methods known in the art. 1-ethyl-2,3,3-trimethyl-3H-indolinium-5-sulfonate was then heated with N,N-diphenylformamidine at 130° C. in acetic anhydride for 1 h and vinylogous product (18) obtained as a solid product after the reaction mixture is treated with diethyl ether.

Preparation of Compound (19). 1-(6-carboxyhexyl)-2,3,3-trimethyl-3H-indolinium-5-sulfonate (19) was synthesized by heating 2,3,3-trimethyl-3H-indole-5-sulfonic acid (1.35 g, 4.84 mmol) and commercially available 6-bromohexanoic acid (1.2 g, 6.15 mmol) in refluxing nitromethane (10 mL) for 24 h, diluting with isopropanol (100 mL) upon cooling to room temperature and collecting the solid precipitate produced after storing at −20° C. in the freezer overnight (1.5 g, 79% yield).

Preparation of Compound (20). Compound (18) (0.704 g, 1.71 mmol), Compound (19) (0.79 g, 2.23 mmol), acetic anhydride (5 mL) and pyridine (5 mL) were heated together in an oil bath at 120° C. for 2 h. The solution was then cooled to room temperature and 100 mL of diethyl ether added to separate out the product. The ether supernatant was removed by decantation and the residual product was washed 2 times with more ether. The product was then taken up in 10 mL of water and purified by C18 reversed phase silica gel chromatography eluting with a 0 to 30% gradient of methanol to water to provide pure material. Material was characterized by ¹H NMR (D20) and shows the following peaks at ppm: 8.30 (triplet, 1H), 7.75 (singlet, 2H), 7.70 (triplet, 2H), 7.20 (triplet, 2H), 6.20 (multiplet, 2H), 3.90 (multiplet, 4H), 2.20 (triplet, 2H), 1.30-1.70 (multiplet, 16H), 1.25 (multiplet, 2H), 1.20 (triplet, 3H)

Preparation of Compound (21) (DPA-NH₂). This compound was prepared in 6 steps via methods known in the art.

Preparation of Compound (22). Compound (20) (32 mg, 0.051 mmol) is dissolved and stirred in anhydrous DMF (1 mL) and 2 drops of dry triethylamine from a 50 μL syringe were added. Solid 2-(1H-benzotriazol-1-yl)-1,1,3,3-tetramethyluronium hexafluorophosphate (HBTU) (24 mgs, 0.063 mmol) was added and the solution was stirred for 2 mins to form the activated ester intermediate, then Compound (21) (31 mg, 0.053 mmol) in anhydrous DMF (1 mL) was added and the mixture was stirred at room temperature overnight. The solvent was removed by rotary evaporation under reduced pressure and the residue was dissolved in a small volume of water and purified by C18 reversed phase silica gel chromatography eluting with a 10 to 30% gradient of acetonitrile to water with 0.1% trifluoroacetic acid added to provide pure apo-Compound (22) (30 mg, 47.2% yield) with observed (M+2H)⁺=1200.7 and calculated (M+2H)⁺=1201.5. A solution of zinc nitrate hexahydrate in methanol (1 mL at a concentration of 15.8 mg/mL; 0.053 mmol) was added to apo-(E) (30 mg, 0.025 mmol) and the solution was stirred for 1 h then concentrated and dried under high vacuum to provide 35 mg of Compound (22). A 1 mM stock solution of Compound (22) in water was prepared.

### Example 3

This example provides a description of compounds of the present disclosure and uses of same.

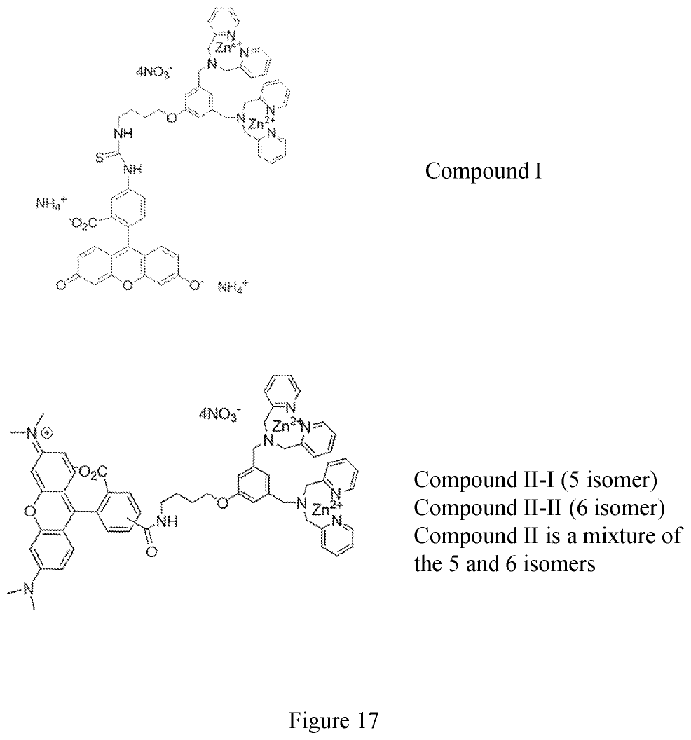

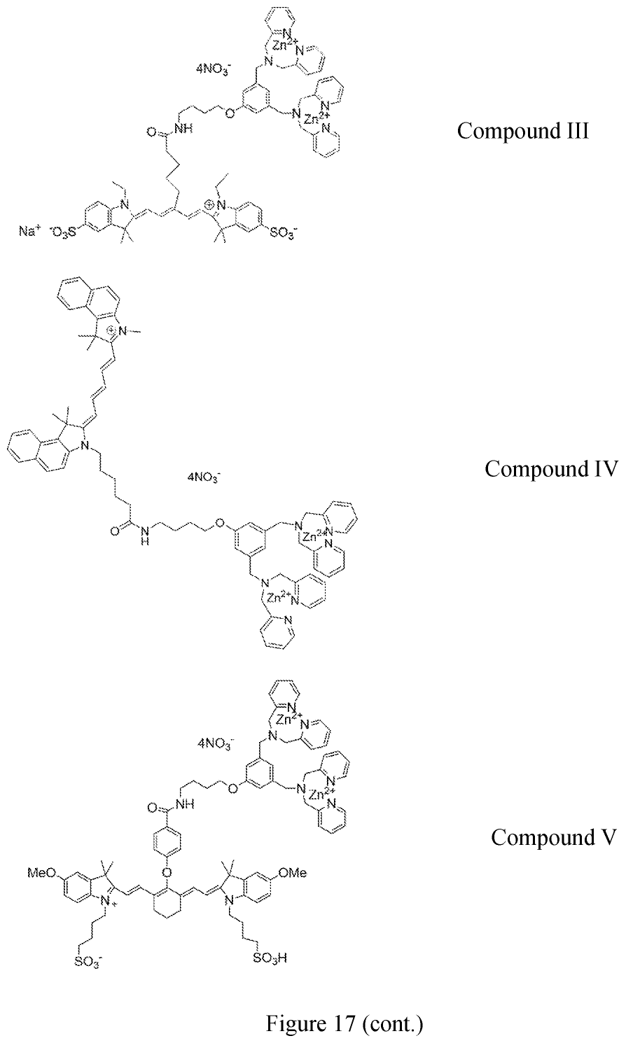

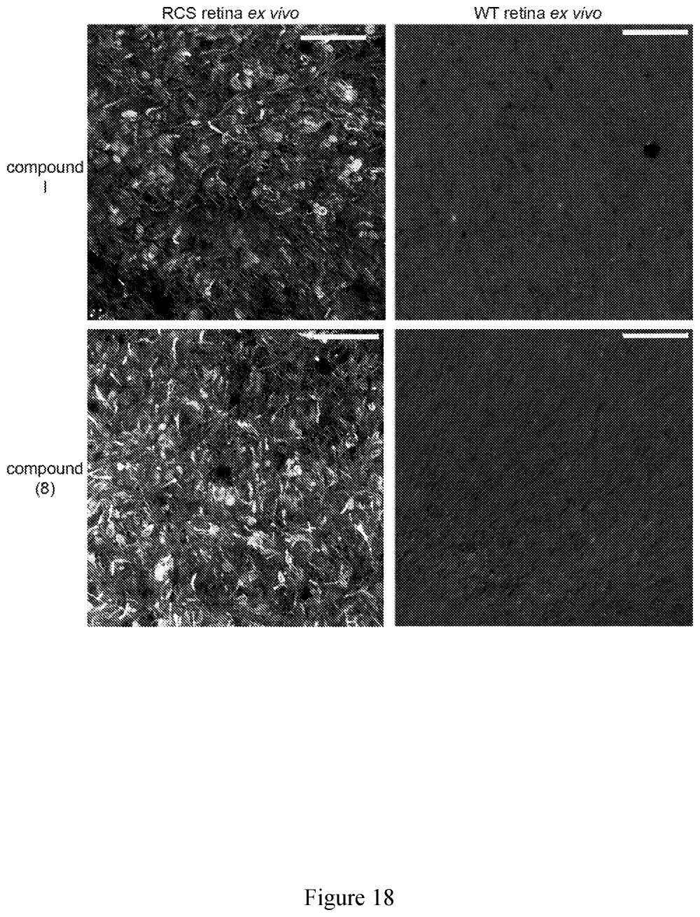

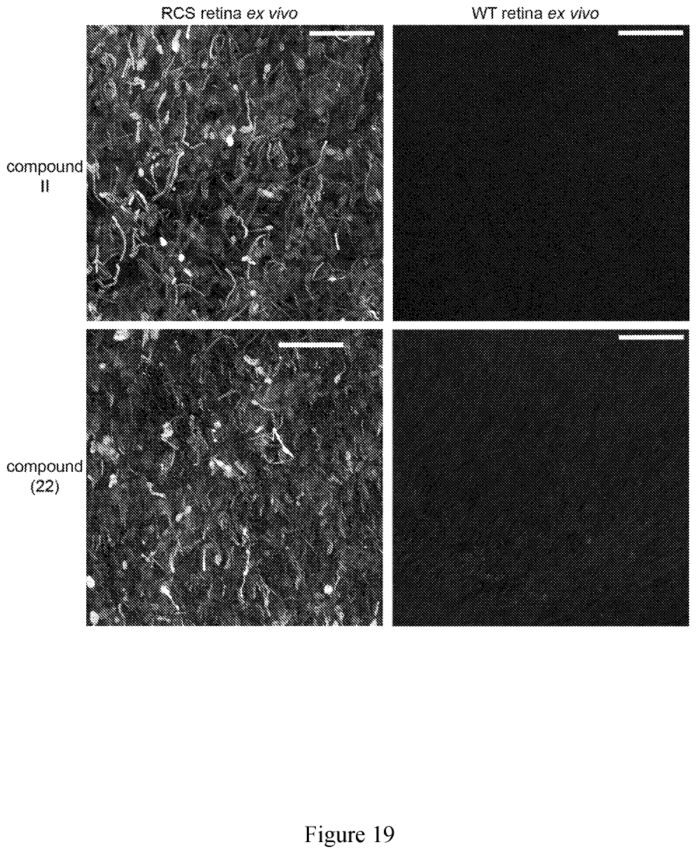

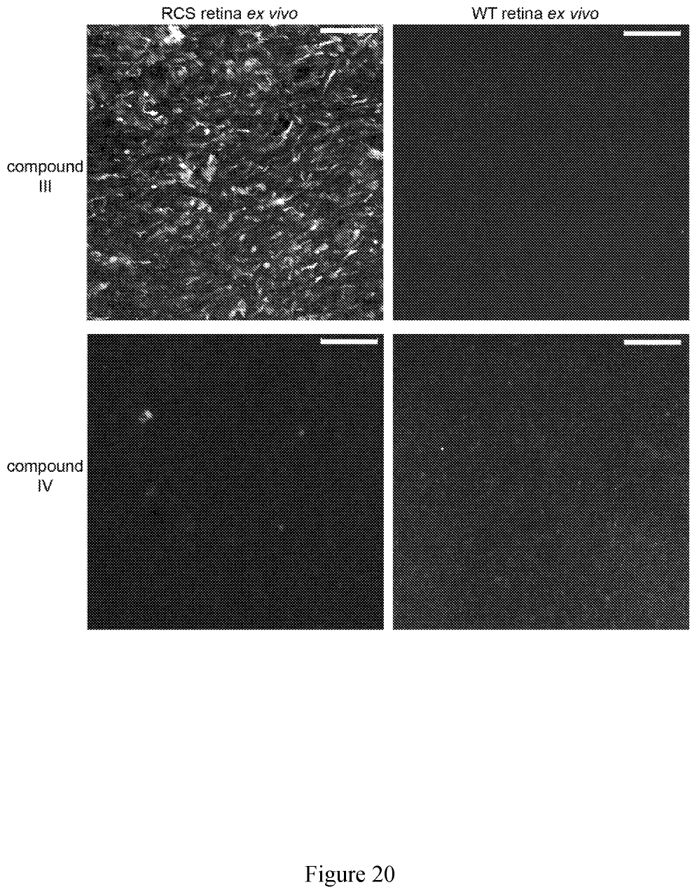

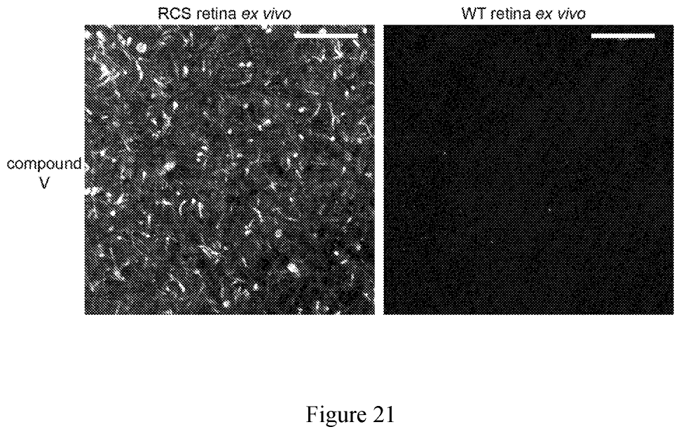

Phosphatidylserine externalization is an early molecular signature for apoptosis. In many retinal degenerative diseases, photoreceptor neurons die by apoptosis. Described herein is the utility of the phosphatidylserine-binding conjugate of Bis(zinc(II)-dipicolylamine (Zn-DPA) with Texas-red (Compound II, a mixture of a compounds II-I and II-II (see FIG. 17)) in transiently labeling apoptotic photoreceptors in living pigmented or albino rats and mice with retinal degeneration. Applying Compound II as an eye drop is non-toxic and eliminates the need for intraocular injection. Compound II fluorescence specifically and transiently labeling dying retinal photoreceptors is detectable in anesthetized animals using standard retinal or whole small animal imaging systems. Importantly, prior Compound II eye drop administration and imaging does not affect repeat testing. Altogether, these results establish Compound II imaging as a completely non-invasive method that provides the opportunity to longitudinally monitor retinal photoreceptor cell death in preclinical studies.

Bis(zinc(II)-dipicolylamine) (Zn-DPA) is a small (1.84 kDa) synthetic compound that binds to anionic phospholipids including PS. Zn-DPA conjugation to fluorophores yields probes that are suitable for PS live imaging. Compound I (like annexin-V-protein probes) administered by intravitreal injection successfully labels dying retinal ganglion cells, the innermost retinal neurons that directly neighbor the vitreous injection site. Utility of non-invasive PS probes in labeling apoptotic photoreceptors, the outermost retinal neurons, has not been reported to date. Described herein is Texas-red-conjugated ZnDPA (Compound II) detects photoreceptor apoptosis in living mice and rats when administered as an eye drop. This procedure avoids intraocular injection, which may itself alter the retinal degenerative process.

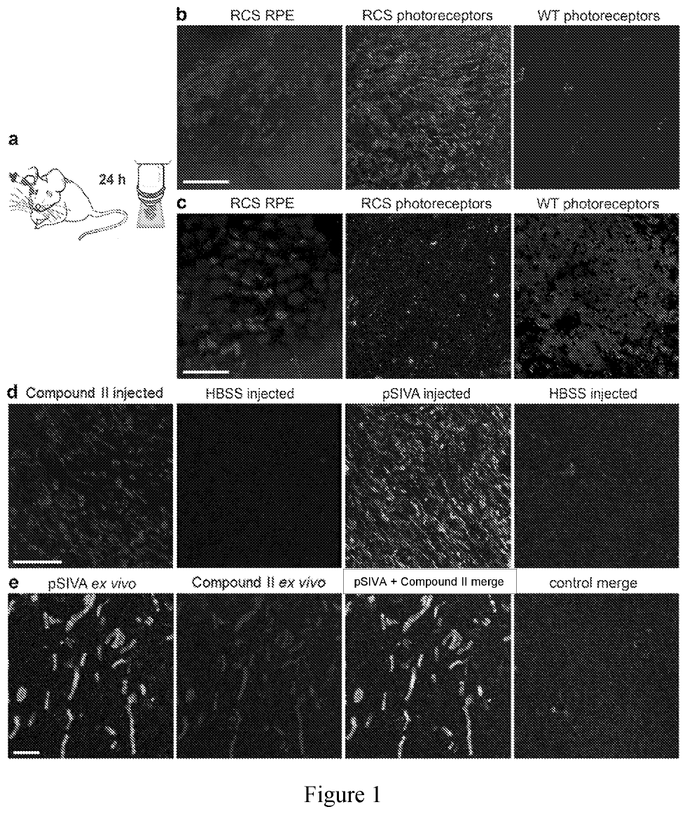

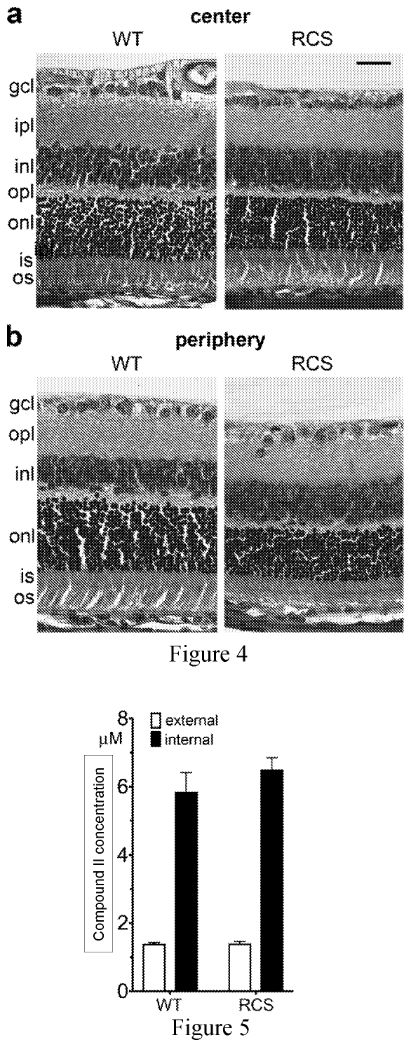

Specific Compound II labeling of apoptotic photoreceptors 24 hours after application as eye drop. To test whether Compound II has utility as in vivo apoptosis indicator, eye penetration was assessed in a well characterized rat model of retinal degeneration, the Royal College of Surgeons (RCS) rat (RCS-rdy-p, pink-eyed). RCS rats lack photoreceptor outer segment renewal due to disruption of the mertk gene, which encodes a key clearance phagocytosis receptor. This results in rapid, synchronized photoreceptor death by apoptosis beginning around postnatal day 25 (p25). Indeed, P25 RCS rats showed intact retinal morphology with conserved inner and outer segments similar to age-matched wild-type (WT) rats (FIG. 4). Thus, p25 rats were explored for Compound II testing. The probe was applied as eye drop to anesthetized RCS and WT rats. Rats were sacrificed 24 hours later, and neural retinas and posterior eyecups were dissected and immediately imaged live, mounted with either photoreceptors or retinal pigment epithelium (RPE) tissue side up (FIG. 1a). Fluorescence was only detected in the neural retina of RCS rats, indicating that Compound II applied to the ocular surface reaches the photoreceptors and specifically labels apoptotic cells (FIG. 1b). To test if Compound II penetrates the eye equally in WT and RCS rats, Compound II was quantified in external rinse (to account for remaining free dye) before opening the eyeball and internal rinse (containing likely mostly vitreous) obtained from the posterior aspect of the eye following removal of the anterior segment 3 hours after eye drop administration. ˜4-fold higher Compound II concentration inside as compared to outside the eye and similar levels of Compound II in WT and RCS rat eyes (P=0.621) showed that eye drop-administered Compound II penetrates the anterior tissues of the eye to reach the posterior eyeball irrespective of retinal degeneration (FIG. 5). Also tested was the utility as eye drop of a fluorescent PS biosensor that is an annexin derivative, “Polarity Sensitive Indicator of Viability & Apoptosis” (pSIVA). However, following pSIVA eye drop application, specific pSIVA fluorescence of RCS apoptotic photoreceptors (FIG. 1c) was not detected. In contrast, when either Compound II or pSIVA were injected intravitreally, both dyes labeled apoptotic photoreceptors in RCS retina and exhibited similar staining patterns (FIG. 1d). Thus, lack of labeling following pSIVA administration as eye drops was likely due to its inability to reach the outer retina. Finally, freshly excised RCS rat retina were co-stained with a mix of Compound II and pSIVA followed by immediate live imaging as flat mount specimen. Both dyes labeled the same cells and structures including those with appearance of outer segments in these ex vivo experiments further supporting the staining specificity of Compound II for apoptotic photoreceptors in the degenerating RCS retina (FIG. 1e).

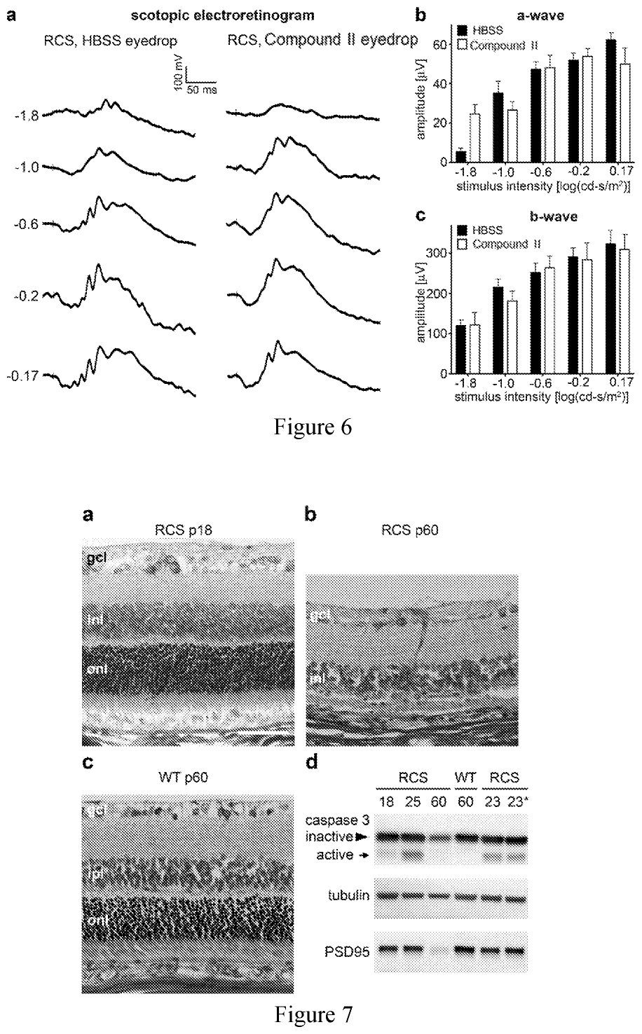

Lack of direct retinal toxicity of Compound I. To test toxicity of Compound II, scotopic full-field electroretinograms was performed on dark-adapted rats. Three days after eye drop application, there was no difference in light responses between RCS rats treated with Compound II eye drops and littermates treated with Hanks buffered saline solution (HBSS) control eye drops (FIG. 6). Thus, Compound II eye drops do not directly affect functionality of retinal neurons or the course of ongoing retinal degeneration.

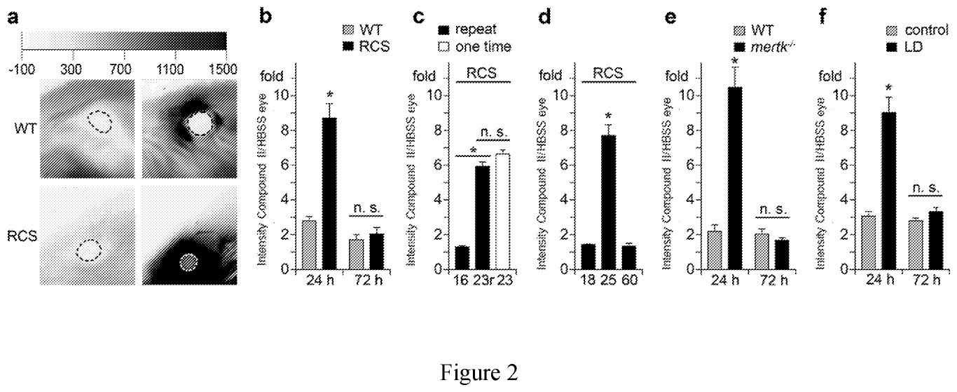

In vivo detection of apoptotic RCS photoreceptors by whole animal imaging. Next, probe fluorescence in eyes of live, anesthetized WT and RCS rats was imaged after application of Compound II to one eye and HBSS control eye drop to the other (FIG. 2a). Fluorescence of contralateral eyes was measured to yield background fluorescence intensity, and Compound II-derived signals were quantified as fold increase over background specific to each animal. Using a whole animal scanner, recording fluorescence of the entire eye 24 hours after Compound II application it was found that fluorescence of RCS Compound II-treated eyes was elevated 8.7-fold (P<0.001), while fluorescence of WT Compound II-treated and contralateral eyes did not differ significantly (P=0.72) (FIG. 2, a and b). Similar experiments using unconjugated fluorophore without the Zn-DPA targeting moiety did not increase the fluorescence signal (data not shown). Fluorescence of Compound II-treated RCS eyes decreased to control levels between 24 and 72 hours such that there was no difference in fluorescence between RCS and WT eyes or between treatments 72 hours after Compound II application (P=0.98) (FIG. 2b). Thus, Compound II eye drops transiently label degenerating RCS photoreceptors in the living eye.

The transitory nature of Compound II labeling was used to repeatedly test the same animal, which will have utility in longitudinal studies. To this end, Compound II eye drops were applied to p16 RCS rats followed by whole animal live imaging 24 hours later. One week after the first eye drop application, the same rats at p23 again received Compound II eye drops followed by imaging the next day. As controls, littermate p23 RCS rats not manipulated previously were also tested. These experiments showed that Compound II eye drops yield negligible labeling in RCS rats at p16, an age prior to onset of apoptosis (FIG. 2c, black bar 16). Repeating the non-invasive Compound II labeling at p23 on the same rats yielded ˜4.5-fold higher Compound II signals compared to the signal detected at p16 (P<0.0001) (FIG. 2c, black bar 23r). Notably, Compound II fluorescence in littermate RCS rats that were tested at p23 alone did not differ from signals in p23 RCS rats that were tested twice (P=0.092) (FIG. 2c, compare black bar 23r and white bar 23). Thus, Compound II eye drop labeling and live imaging allows longitudinal testing of the same animals on a weekly basis.

To further confirm that Compound II labels only RCS photoreceptors that are in the process of undergoing apoptosis, RCS rats were tested at different stages of disease. Normal morphology of RCS rat retina at p18 and complete loss of photoreceptors by p60 (FIG. 7, a-c) was confirmed. Caspase-3 immunoblotting further showed cleaved, active caspase-3 indicative of ongoing apoptosis at negligible levels at p18 and p60 as compared to robust levels at p25 (FIG. 7d). Direct comparison of RCS rats at p18, p25, and p60 in Compound II non-invasive imaging showed elevated signal as before at p25 while p18 and p60 signals were not significantly elevated and not significantly different from each other (P=0.951). These results show that the methods of the present disclosure label RCS rat retina only at an age with active apoptosis but not before onset of apoptosis or after photoreceptors are lost.

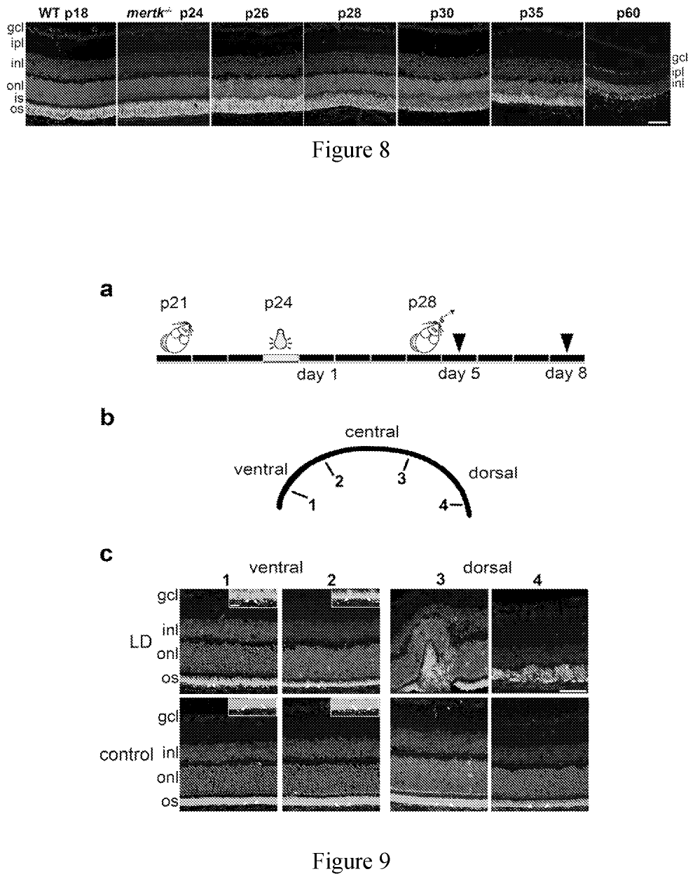

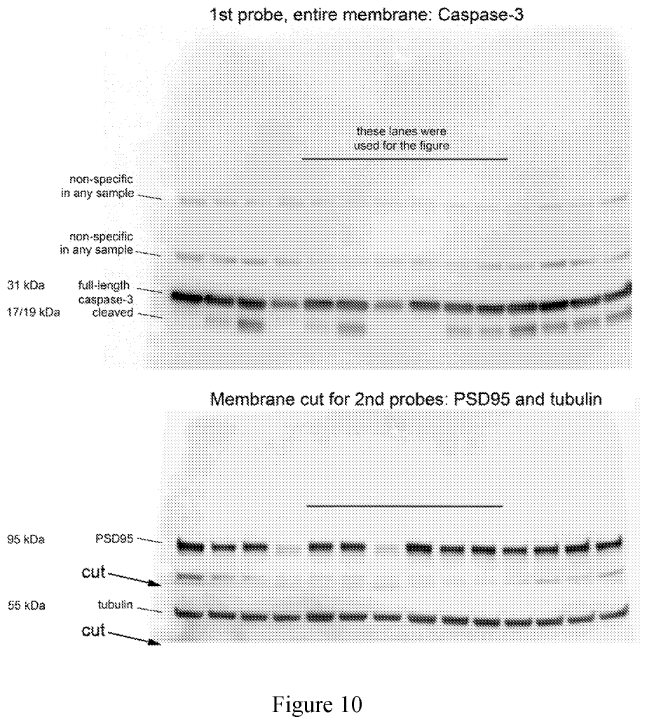

In vivo detection of apoptotic photoreceptors in pigmented mertk^{-/-} mice. Like RCS rats, mertk^{-/-} mice lack expression of the clearance receptor MerTK. Pigmented mertk^{-/-} mice that had been backcrossed extensively to 129SvEms/J WT mice were studied. As genetic background affects the retinal phenotype of mertk^{-/-} mice, it was first determined the time course of retinal degeneration in this strain (FIG. 8). p28 mertk^{-/-} mice retained mostly normal retinal morphology and were thus chosen for this study. Live whole eye imaging performed 24 hours after Compound II eye drop application measured a 11.2-fold increase in fluorescence in Compound II-treated mertk^{-/-} eyes compared to contralateral eyes (P=0.0038), while no probe-specific increase was detected in strain- and age-matched WT eyes (P=0.87) (FIG. 2e). Like in RCS retina, Compound II signals in mertk^{-/-} retina decreased to background levels by 72 hours after eye drop application (FIG. 2e). These results show that Compound II eye drops allow live imaging of photoreceptor degeneration in rats and in mice, and, importantly, that pigmentation of eye tissues does not preclude detection of Compound II signals emitted from the outer retina.

In vivo detection of apoptotic photoreceptors in WT rats following light damage. In both RCS rats and mertk^{-/-} mice, debris of degenerating photoreceptors accumulates in the subretinal space due to defective RPE clearance phagocytosis forming a debris zone. Such debris may expose PS and/or bind Compound II yielding a larger PS signal from Compound II eye drops than in other forms of retinal degeneration where photoreceptor apoptosis is accompanied by clearance of dead or dying photoreceptors or their debris. Detection of dying photoreceptors following light injury of WT rats whose RPE has intact clearance activity was therefore tested. Bright white light exposure triggered acute retinal degeneration as expected with early stage of retinal degeneration five days after light damage (FIG. 9). Compound II eye drops were thus administered to control and light-damaged (LD) rats four days after light exposure followed by live imaging 24 hours later. Live whole animal scanning detected a 9-fold increase in Compound II fluorescence in eyes of LD rats compared to contralateral eyes (P<0.001), while no compound-specific increase was detected in control animals (P=0.95) (FIG. 2f). As in the MerTK-deficient models, fluorescence intensity decreased to background levels by 72 hours after Compound II application (FIG. 2f). These results demonstrate that PS exposure by photoreceptors is detectable using non-invasive live Compound II imaging in early ongoing retinal degeneration induced acutely in LD WT rats with normal RPE debris clearance activity.

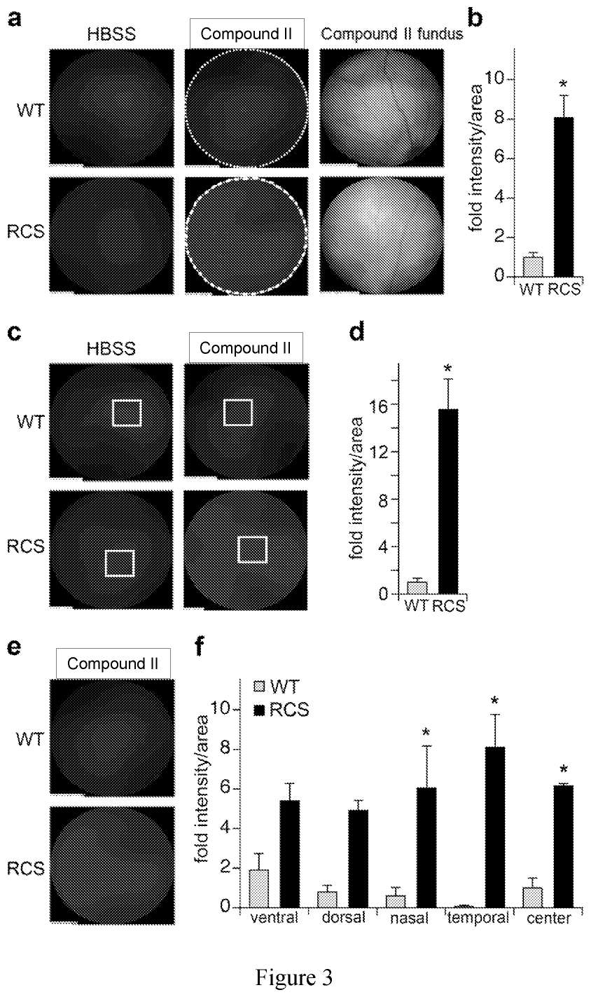

In vivo detection of apoptotic RCS rat photoreceptors by retinal imaging. Small animal fluorescence imaging instruments are widely available to the scientific community and do not require expertise in vision science. However, specialized retinal imaging systems allow focusing on specific eye tissues. Compound II eye drop treatment of p25 RCS and WT rats was tested followed by fluorescence detection using a retinal imaging system. Fluorescence imaging at the site of degenerating photoreceptors detected an 8.6-fold increase in Compound II-treated RCS eyes over contralateral eyes (P=0.0035) while WT eyes did not differ significantly (P=0.29) (FIG. 3, a and b). Fundus images were also recorded that confirmed known changes in retinal blood vessels in degenerating RCS retina (FIG. 3a). Retinal imaging further allowed quantification of separate retinal regions, which is meaningful as many forms of retinal degeneration progress with characteristic topography. In RCS eyes, the maximal Compound II-specific fluorescence was 15.4-fold as intense as the maximal non-specific fluorescence in the contralateral eye (P=0.004) (FIG. 3, c and d). Compared to Compound II-treated WT eyes, Compound II-treated RCS eyes showed increased fluorescence in all quadrants, but changes in the temporal quadrant were most pronounced and only nasal, temporal, and central regions were statistically different from WT (FIG. 3, e and f) (inferior (ventral) P=0.318, superior (dorsal) P=0.149, nasal P=0.022, temporal P=0.004, center P=0.003). These findings were in agreement with histology studies showing that RCS photoreceptor death progresses from the center to the periphery.

The results described herein establish non-invasive in vivo detection in retinal photoreceptor neurons of PS exposure, the cardinal feature of early apoptosis. The method described herein requires only a one-time application of the non-toxic, Compound II as an eye drop. PS signal detection by live imaging succeeds in three well established rodent animal models of retinal degeneration testing mice and rats, pigmented and albino animals, and inherited and induced retinal degenerative models was shown.

It was determined that the small chemical compound Compound II, but not the annexin protein based pSIVA labels dying photoreceptors following application as eye drop. Annexin V, the most widely used probe for PS exposure, was not tested but it is unlikely to be useful as eye drop given its structural similarity to pSIVA. All three PS probes label dying photoreceptors in the models explored herein when applied ex situ. Here, it was shown that pSIVA and Compound II yield similar staining of apoptotic outer retina in RCS rats following intravitreal injection and co-stain apoptotic RCS photoreceptors ex situ. Thus, it is believed lack of labeling by pSIVA following eye drop application is due to its failure to reach the photoreceptor layer of the retina possibly due to its molecular size, which is larger than Compound II.

To generate the data set presented, Compound II was used as it matched well the excitation/emission settings of the whole animal fluorescence scanner used. The live imaging experiments consistently showed similar levels of background fluorescence in eyes not receiving Compound II with and without retinal degeneration independently of the animal model tested. In non-degenerating retina, Compound II eye drops elevated fluorescence slightly but these increases did not reach statistical significance in any of the models tested. This was not due to lack of Compound II penetration into ocular tissues in non-degenerating retina, which were directly tested to be equal in WT and RCS rats. Altogether, these results imply little interference from naturally fluorescent molecules in the eye/retina at the excitation/emission setting used to image Compound II well justifying its use.

In vivo imaging of Compound II was performed 24 hours after eye drop application. Detection of the dye in the posterior eye by 3 hours after eye drop application indicates that imaging for detection may succeed at earlier time points. However, at least for longitudinal studies 24-hour intervals in between anesthesia of individual animals may be advisable. By 72 hours after Compound II eye drop application, specific Compound II fluorescence was no longer detectable in all three animal models indicating that Compound II is completely cleared from the outer retina between 24 and 72 hours after application although retinal degeneration continues to progress. These findings are consistent with those from Compound II imaging in rodent models of myopathy and ischemia-reperfusion where Compound II was administered systemically. The rapid clearance may be considered rather advantageous as it minimizes the chance that the probe alters and possibly worsen the course of retinal degeneration. In support, no effect of Compound II eye drops on retinal function as tested by ERG 72 hours after application was found. Moreover, the loss of applied Compound II within 72 hours allowed re-application and repeat measurements of the same animal during progressive forms of retinal degeneration. Importantly, it was established specifically that re-testing the same animal one week after the initial testing was successful and that prior testing did not affect results of the repeat testing. Altogether these data demonstrate that Compound II eye drops will have utility for longitudinal studies.

In this work, establishing non-invasive detection methodology for three different animal models that share a pan-retinal degeneration of photoreceptor neurons was described. However, given penetration of eye drop-administered Compound II irrespective of retinal degeneration, it is considered that this method may be adapted to non-invasive detection of apoptosis by other retinal neurons as well. It is promising in that respect that detection of early apoptotic retinal ganglion cells following intravitreal Compound I injection has already been successful in dissected, fixed tissue in a rat model of induced excitotoxicity. In addition, retinal imaging may succeed in detecting focal regions of cell death for instance following acute laser injury although there will surely be a minimum size of damaged retinal region and extent of photoreceptor apoptosis for detectability.

PS exposure is a universal characteristic of early apoptotic cells and apoptosis is common to many if not most retinal diseases involving death of photoreceptors. These results show that Compound II imaging detects photoreceptor death in small rodent animal models. Such small animal models may be raised in sufficient numbers to afford testing multiple cohorts in analyses requiring animal sacrifice. However, besides the inherent value of reducing the overall numbers of animals needed for research non-invasive survival Compound II imaging of retinal cell death will additionally be of significant scientific value. Longitudinal studies will be able to identify periods of cell death during development of retinal degeneration thereby allowing selection of specific informative animal ages for the in-depth histology studies. As PS exposure precedes irreversible changes during cell death, anti-death therapies may be tested at the time of Compound II fluorescence detection for efficacy in delaying retinal degeneration. Preselecting animals for experimental therapies based on Compound II imaging will likely lessen data variability resulting from the use of animals based on ages alone without accounting for animal to animal variability in age of onset or speed of progression of retinal degeneration. Finally, Compound II imaging does not require specific eye research expertise or equipment. The presence of either a retinal camera or a small animal imager is common to academic and industry research labs. Compound II imaging offers a quick and economical approach to pre-screening any of the vast numbers of rat and mouse animal models that continue to be generated for the non-eye research in interdisciplinary collaborations.

This study demonstrates no differences between mice and rats with respect to Compound II detection of apoptotic retinal photoreceptors.

In conclusion, these results establish Compound II eye drop application and imaging as safe, completely non-invasive approach to monitoring photoreceptor death in small animal models of retinal degeneration. Given its simple protocol and the transient nature of Compound II labeling allowing repeat testing, it is considered that this method will be widely applicable and useful for the field.

Materials and Methods. Reagents were purchased from Thermofisher (Carlsbad, Calif.) or Millipore-Sigma (St. Louis, Mo.) unless otherwise indicated.

Animals. Animals of both sexes were used. Pink-eyed dystrophic RCS rats (rdy/rdy-p) and Sprague Dawley (SD) wild-type (WT) albino rats were bred and raised to yield litters at defined age for experiments. mertk^{-/-} mice in 129Sv background were raised by crossing B6-129-Mertk^{tm1Grl}/J (Jackson Laboratories, strain #11122) for 9 generations with 129T2/SvEms/J (Jackson Laboratories strain #2065) WT mice. mertk^{-/-} mice were genotyped using published protocols and were found not to carry the rd8 mutation. WT 129T2/SvEms/J mice were raised as controls.

Animals were housed under cyclic 12 h:12 h light-dark conditions with food and water ad libitum. Animals were housed in metal racks in non-transparent cages with metal lid supporting both water bottle and food pellets. This configuration provided sufficient shielding to minimize illumination inside cages. Light intensity varied from 10 lux in the back of the cage to 60 lux in front.

Anesthesia was induced by intraperitoneal injection of a mix of 100 mg/kg ketamine and 10 mg/kg xylazine.

Compound II dye preparation and fluorescent probe eye drop application. Compound II was obtained from Molecular Targeting Technologies Inc. (West Chester, Pa.) and reconstituted according to the manufacturer's instructions. The resulting 1 mM solution of Compound II was stored in the dark at 4° C. and used within 14 days directly as eye drop. 6-7 hours after light onset, anesthetized animals received in both eyes one drop of 0.5% proparacaine hydrochloride (Akorn, Lake Forest, Ill.) for 2 min as local anesthetic followed by one drop of 2.5% phenylephrine (Akorn) for 2 min to yield eye protrusion. This was followed by one 15 μl drop of 1 mM Compound II solution or of pSIVA solution as provided by the manufacturer (Novus Biologicals, Littleton, Colo.) in HBSS while the contralateral eye received one eye drop HBSS as control for 15 min. The eye drop volume was chosen such that the eye cavity was completely filled without spillage outside the eye, and it may need to be modified depending on eye size. Compound II at 0.5 mM was also tested initially on p25 RCS and WT rats with similar results. The 1 mM Compound II concentration was used for all studies such that probe availability would not be limiting for labeling. Rats were kept in darkness overnight before sacrifice and tissue harvest after 24 hours from the application.

Compound II dye penetration testing. P25 RCS and control WT rats received Compound II or HBSS control vehicle as eye drops under anesthesia and as described above. Animals were kept in the dark for 3 hours before sacrifice. The eyeball was rinsed with 10 μL HBSS to capture any remaining external probe, this rinse was analyzed as external sample. Following lens removal 10 μL HBSS was pipetted into the posterior eyeball, the re-collected liquid was analyzed as interior sample. Cleared samples were analyzed by dot blotting on nitrocellulose membrane followed by quantification of probe fluorescence intensity with a Sapphire Biomolecular Imager (Azure Biosystems, Dublin, Calif.) and direct comparison against a serial dilution of 1 mM Compound II stock applied to the same membrane.

Intravitreal injections. p24 WT and RCS rats were anesthetized. Using a dissecting microscope, 4 μL of 1 mM Compound II or of pSIVA as provided by the manufacturer were injected into the right eye vitreous via the transscleral route using a SilFlex tubing and holder driven by a 10-μL glass NanoFil™ microsyringe (World Precision Instruments, Sarasota, Fla.). An identical volume of HBSS vehicle was injected into the left eyes of the same animals. Animals were maintained in the dark until sacrifice and tissue harvest.

Tissue dissection, live imaging, and fixed tissue histology. For tissue harvest, animals were sacrificed by CO₂ asphyxiation followed by immediate eye enucleation and dissection.

For live imaging, neural retina and posterior eyecup tissue containing the RPE were dissected and separately mounted live in HBSS for immediate imaging on a TSP5 laser scanning confocal microscopy system (Leica, Mannheim, Germany). For ex vivo labeling, freshly dissected retinas from untreated rats were mounted in HBSS containing 2 μM Compound II and 1/50 pSIVA followed by immediate live imaging. Single dye-labeled samples were tested as controls for channel-to-channel bleed-through.

In each assay, all tissues were imaged using identical settings and compiled and processed identically using Photoshop CS4.

For fixed tissue analyses, enucleated eyes were immersion-fixed in Davidson's fixative followed by paraffin embedding and microtome sectioning, rhodopsin and nuclei counterstain labeling of sections. For morphology analyses, tissues were stained with hematoxillin/eosin (H/E) according to standard procedures. Image stacks representing equal thickness were acquired using equal settings and collapsed to yield maximal projections of center and peripheral retina. Images were recompiled using Photoshop CS4.

Whole small animal fluorescence scanning. Animals were anesthetized, and eyes treated with one drop of 2.5% phenylephrine for 2 min followed by one drop of 1% tropicamide (Akorn) for 2 min for pupil dilation. Animals were placed inside a Kodak FX-Pro imager (Bruker Bioscience Corporation, Billerica, Mass.) with one eye imaged at a time. Animals were imaged using 550 nm light excitation and 600 nm emission. Image analysis was performed with Multispectral FX-Pro software (Bruker). Fluorescence intensities in selected regions of interest (ROI) corresponding to each eye were quantified and the resulting values were compiled as fold intensity Compound II-treated eye over contralateral control eye that received only HBSS as eye drop.

Retinal imaging. 24 hours after receiving Compound II eye drops or control solvent eye drops on one eye and 4 to 6 hours after light onset rats were anesthetized. Age-matched RCS and WT cohorts comprising 3 rats each were tested side-by-side. A Micron-IV retinal camera was used (Phoenix Technology Group, Pleasanton, Calif.). Eyes to be imaged were covered with artificial tears and GONAK (both Akorn) before adjusting the position of the rat's eye such that the camera's eyepiece touched the cornea. 550 nm or white light illumination were used to acquire fluorescent and bright field fundus images, respectively. Images were acquired at different focal planes starting from the cornea to the back of the eye corresponding to the photoreceptor-RPE interface. Fluorescence intensities were quantified in selected ROI using Image J (National Institutes of Health, Bethesda, Md.). Intensity values from contralateral untreated eyes were used to calculate fold intensity change in the Compound II treated eye. For retinal quadrant analyses, data were normalized to the average of the center area of control samples.

Electroretinography. Animals were dark-adapted overnight before recording scotopic responses under anesthesia and under dim red light exactly as described previously using a UTAS-E2000 visual electrodiagnostic system (LKC Technologies, Gaithersburg, Md.). Stimuli were presented in order of increasing intensity as a series of white flashes of 1.5 cd-s/m² attenuated to yield intensities from −1.8 to 0.2 log cd-s/m². For each flash intensity, three to six recordings were averaged. For all recordings, a-wave amplitudes were measured from the baseline to the trough of the a-wave, and b-wave amplitudes were measured from the trough of the a-wave to the peak of the b-wave.

Light damage (LD) induction. Light damage was induced. p21 SD WT rats were maintained in the dark for 65 hours starting 5 hours after light onset before anesthesia and exposure for 1 hour to 10,000 lux of cool-white fluorescent light (Snap-on, 25 W LED Work-Light, broad spectrum light 380-760 nm). This was followed by 23 hours of exposure of the cage to 6000 lux using the same light source at greater distance with animals having access to food and water ad libitum and moving freely. Air temperature in the cage was monitored with an infrared thermometer (Tempgun TG1, NY) and maintained below 23° C. for the entire light exposure period by placing the cage into an open hood with ventilation. Following light exposure, animals were maintained in the dark before experiments. Age-matched control rats were maintained in the same environment and dark adaptation conditions except in normal room light during the 24 hours of bright light exposure.

Sample lysis and immunoblotting. Whole eyes without the lens were lysed in 50 mM Hepes, pH 7, 150 mM NaCl, 10% glycerol and 1% Triton X100 freshly supplemented with protease inhibitor cocktail. Cleared lysates were boiled with reducing SDS sample buffer before separation on SDS-polyacrylamide gels and nitrocellulose membrane transfer using standard protocols. Membranes were sequentially incubated with primary and appropriate horseradish peroxidase-conjugated secondary antibodies and chemiluminescence reagent (Kindle Biosciences) followed by digital imaging using a Kwikquant Imager (Kindle Biosciences). Primary antibodies used were: caspase-3 (#9662, Cell Signaling, Danvers, Mass.), α-tubulin (#2125, Cell Signaling), and PSD95 (#MAB1598, Millipore-Sigma).

Statistical analyses. Statistical analysis was performed by unpaired Student's -test using Microsoft Excel to compare any two samples or by one or two-way ANOVA followed by Tukey's post-hoc test using Prism Graphpad 7.0 for comparison of multiple samples (LaJolla, Calif.). P values<0.05 were considered a statistically significant difference.

Ethical approval and informed consent. All experimental procedures were reviewed and approved by the Fordham University Institutional Animal Care Committee and complied with the policies and regulations regarding animal experimentation. They were conducted in accordance with the National Institutes of Health Guide for the Care and Use of Laboratory Animals (8^{th} ed.) and the ARVO Resolution for the Use of Animals in Ophthalmic and Vision Research.

### Example 4

This example provides a description of the expected characterization of compounds of the present disclosure.

To screen and validate water-soluble compound formulations for sensitivity and tolerance in repeated, non-invasive detection of photoreceptor (PR) death in an extensively characterized model of inherited RD, the RCS rat. Unbiased quantification of sensitivity and safety of new water-soluble compounds for apoptotic PR detection will be performed. Preliminary data shows imaging detection of apoptotic PR as soon as 3 h post eye drop. The eye drop application/imaging protocol will be optimized to shorten the time between application and imaging to decrease wait times for patients thus improving ease of use. Following longitudinal dye testing, ERGs and histology analyses will serve to reveal effects of repeat compound eye drop testing on RD.

Methods and experimental design: Optimization of compound application and in vivo PR death detection protocol: The preliminary data on compound (8) (FIG. 11) will be extended to test whether PR imaging is possible at times <3 h after eye drop application. To this end, 3 groups of 6 RCS rats will be imaged 1, 2, and 3 h after eye drop application. The relative fluorescence signals of eyes treated with compound over water eyes will be calculated to determine the time interval for most effective apoptotic PR detection, which will then be used for all further testing in the proposed study.

Longitudinal testing of compound application and imaging for 6 novel compounds: For each dye, 6 RCS and 6 WT rats will receive eye drops followed by optimized imaging at P18, P25, and P32. Studies will start with dye (8) (FIG. 11), which will be available in sufficient quantity at the start of the project. The 5 other novel compounds will be tested as they become available. In each test experiment, 4 mock treated RCS and WT rats will also be included as negative controls. Dye application and imaging using the MicronIV retinal camera will be conducted as published. Dye will be applied at 1 mM as eye drop to one eye with the contralateral eye receiving water as a negative control. As maximal PR apoptosis and thus labeling in RCS rats is at P25, rats at P28 (without providing eye drops) will also be imaged to determine dye clearance. Fluorescence ratios of contralateral eyes will be calculated. Ratios will be interpreted following decoding of rats. Dyes will be considered useful for future clinical development if they fulfill the following characteristics indicative of specifically and reversibly labeling apoptotic PR in RD:
>4.0 ratio treated to untreated RCS rat eye at 1-3 hours
<1.5 ratio treated to untreated WT rat eye at 1-3 hours
<1.5 ratio treated to untreated RCS rat eye at P28 following eye drop at P25

Testing effects of repeat compound eye drops and imaging on RD and healthy retina. 1 week after completion of the longitudinal dye testing or mock treatments as described above, retinal light responses in RCS and WT rats at P39 will be tested in standard scotopic ERGs. Investigators recording ERGs will be blinded to prior eye drop treatments. 1 week after ERG testing rats at P46 will be sacrificed followed by eye paraffin embedding/sectioning and morphometry analysis of thickness of retina layers in H&E stained sections. Notably, at test ages, RCS rats retain sufficient ERG response and retinal neurons that additional disruption will be quantifiable.

To determine adverse effects of water-soluble compounds in vitro in a human tissue model relevant to cornea epithelium. Rationale: In anticipation of future clinical studies toxicity of the new optimized compounds will be tested on in vitro epithelium tissue prepared from normal human keratinocytes that models the cornea epithelium.

Methods and experimental design: Testing direct toxicity in a human tissue model of cornea epithelium as an indicator for ocular irritation. The novel compounds will be screened for eye irritation potential using the EpiOcular eye irritation test (EIT) (MatTek Corp, MA) to identify any compounds with adverse corneal reactions and avoiding exposing human volunteers to potentially irritating materials during clinical testing. EpiOcular has been used for many years by industry as non-animal alternative to determine Draize scores and to assess mildness/ultra-mildness (sub-Draize) of materials contacting eyes. The EpiOcular test construct is a non-keratinized epithelium prepared from normal human keratinocytes that models the cornea epithelium. Test “tissue” is prepared on porous membrane support allowing application of test compounds on differentiated epithelium directly. The MTT viability assay determines the time of exposure needed for a test article to reduce epithelial viability by 50% (ET-50). Based on the ET-50, the test article is categorized into one of 4 classifications ranging from non-irritating to severe/extreme, which correspond to groupings of Rabbit Draize Eye Scores (MMAS). 18 samples (6 dyes at 3 concentrations) will be sent to MatTek for testing and analysis. Samples will be evaluated with positive and negative controls for irritation (n=3 tissues) and by histology (n=2 tissues). H&E stained histological cross-sections will be evaluated by microscopy to provide an independent assessment of the morphological effects of the test compounds on the EpiOcular tissues. For compounds of the present disclosure, fluorescence reading of compounds of the present disclosure at appropriate ex/em will be recorded to determine whether dye is retained by the cells. This is expected to be a sensitive indicator of cell distress (eliciting PS externalization).

### Example 5

This example provides a description of the use of compounds of the present disclosure.

TABLE 2
Dyes tested.

| | in vivo | ex vivo | enucleated |
|---|---|---|---|
| | whole | retina | whole |
| | animal | confocal | eyeball |
| Tested dye | scanner | microscopy | scan |
| Compound I | Not | X | X |
| | Conclusive | | |
| Compound (8) | X | X | not tested |
| Compound II (a mixture of | X | X | X |
| isomers Compounds II-I | | | |
| and II-II) | | | |
| Compound (22) | X | X | X |
| Compound III | X | not | X |
| | | conclusive | |
| Compound IV | did not yield good signal | | |
| Compound V | X | X | not tested |

All experiments were conducted as described in Mazzoni et al. 2019. In brief, RCS and WT rats received eye drops with single compounds. After 3 to 24 hours rats were anesthetized and scanned for ocular fluorescence in a whole animal scanner (Kodak in vivo pro). Subsequently, rats were sacrificed followed by eye enucleation, scanning of whole eyeballs in the animal scanner to determine whether there was interference from extraocular tissues, followed by retinal dissection and ex vivo flatmount retina confocal microscopy photoreceptor side up to directly image fluorescent compound that had penetrated the eyeball and stained apoptotic photoreceptors. Signals were interpreted as positive if fluorescence of compound eye drop treated eye was significantly elevated in RCS retina as compared to WT retina.

Although the present disclosure has been described with respect to one or more particular embodiments and/or examples, it will be understood that other embodiments and/or examples of the present disclosure may be made without departing from the scope of the present disclosure.

1. A compound having the following structure: 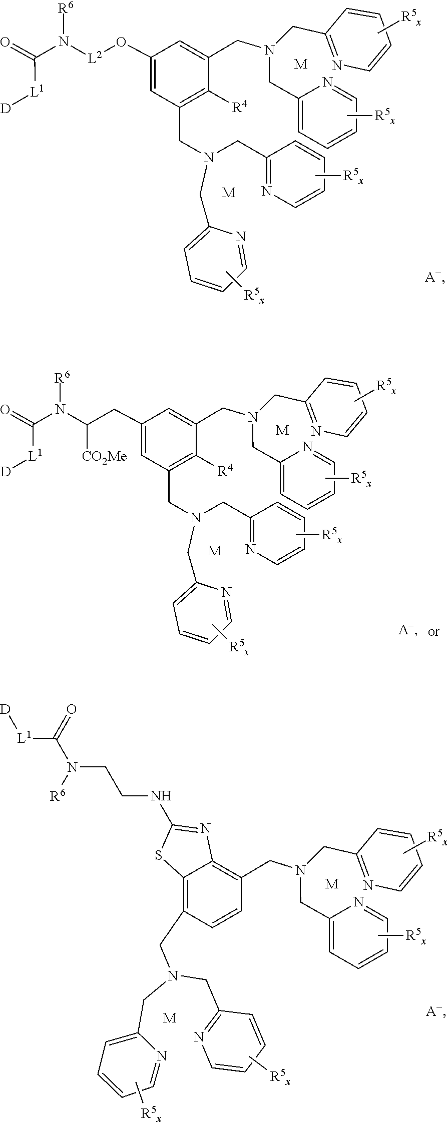
wherein
D is chosen from: 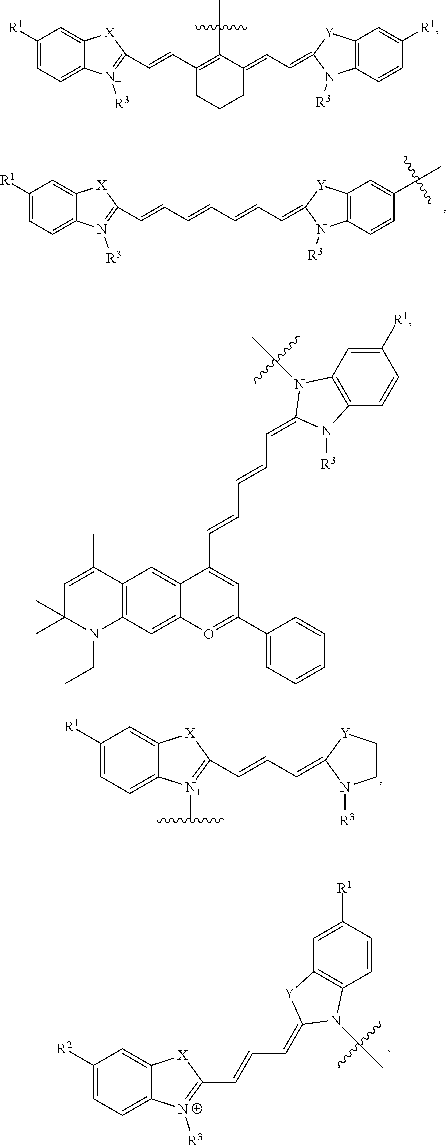 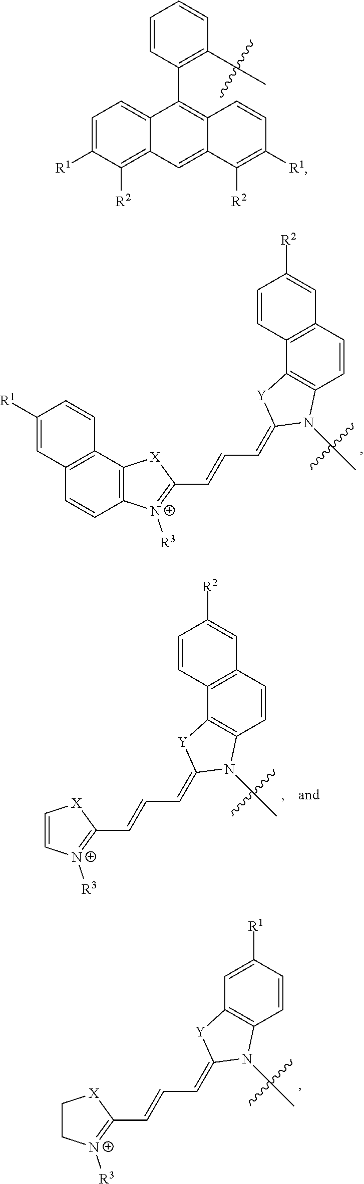
R¹ and R² are polar groups that may be the same or different;
R³ is chosen from alkyl groups, polyethylene glycol groups, and C₁ to C₁₂ alkyl sulfonic acid groups;
R⁴ is chosen from —H and —OH;
R⁵ is chosen from —H, alkyl groups, amine groups, amide groups, carbamide groups, carbamate groups, and halogen groups;
R⁶ is chosen from —H and alkyl groups;
L¹ and L² are independently optional linking groups that may be the same or different;
A is one or more counterions;
M is a divalent cation;
x is individually at each occurrence is 1-4;
Z is C(R₆); and
X and Y are independently C(R⁶)₂, O or S, where each R⁶ is the same or different.

2. The compound of claim 1, wherein the polar groups are individually chosen from protonated sulfonic acid groups, deprotonated sulfonic acid groups, C₁ to C₁₂ alkyl sulfonic acid groups, polyethylene glycol groups, sugar groups, quaternary ammonium groups, phosphonic acid groups, salts thereof, and combinations thereof.

3. The compound of claim 1, wherein linking groups are chosen from ethyl groups, propyl groups, butyl groups, pentyl groups, hexyl groups, PEG having to 2-6 repeat units, and combinations thereof.

4. The compound of claim 1, wherein the divalent cations are chosen from Mn²⁺, Fe²⁺, Co²⁺, Ni²⁺, Cu²⁺, Zn²⁺, and combinations thereof.

5. The compound of claim 1, wherein the compound has the following structure: 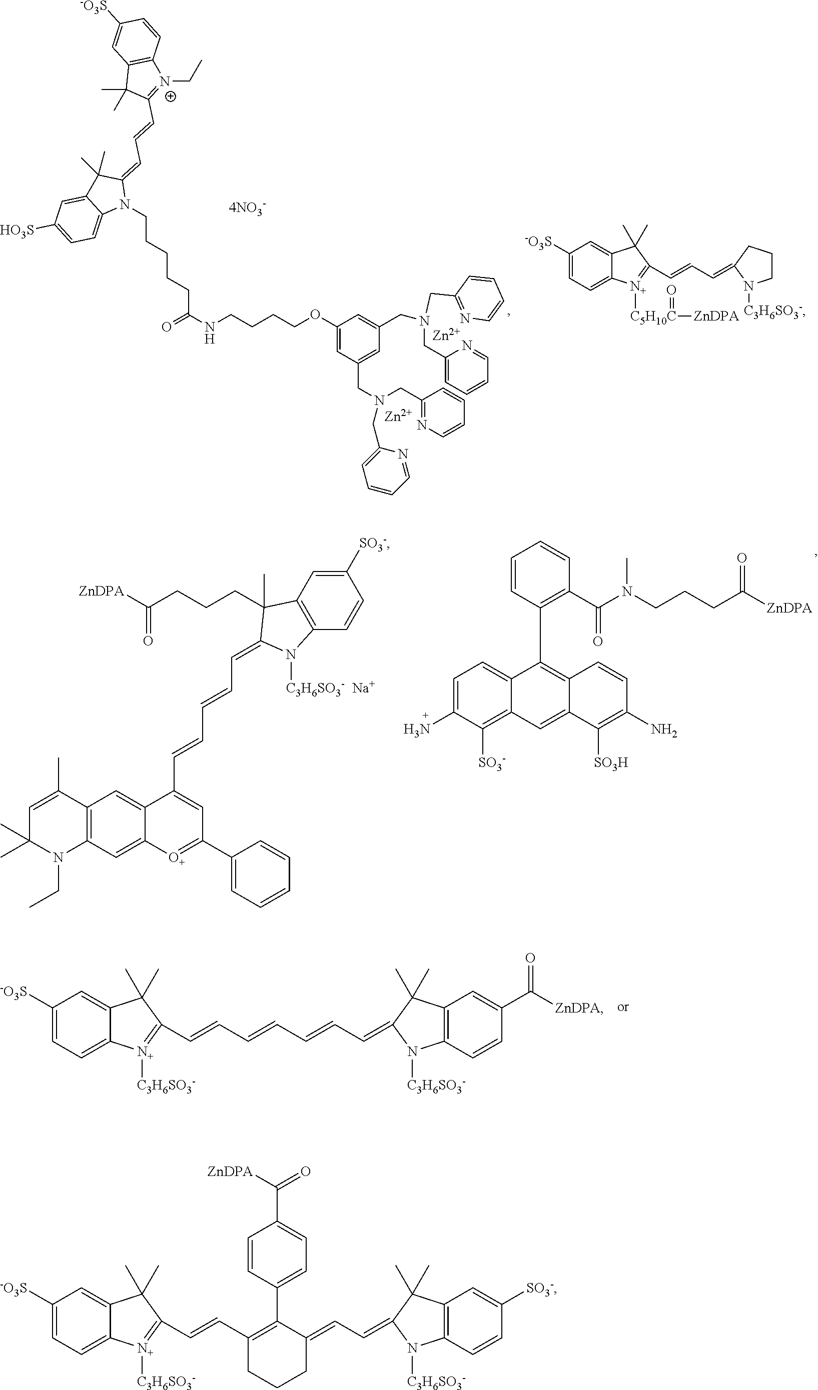
wherein ZnDPA has the following structure:

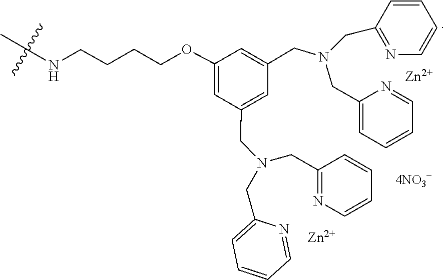 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

6. A composition comprising a compound of claim 1 and a pharmaceutically acceptable carrier.

7. The composition of claim 6, wherein the compound has the following structure: 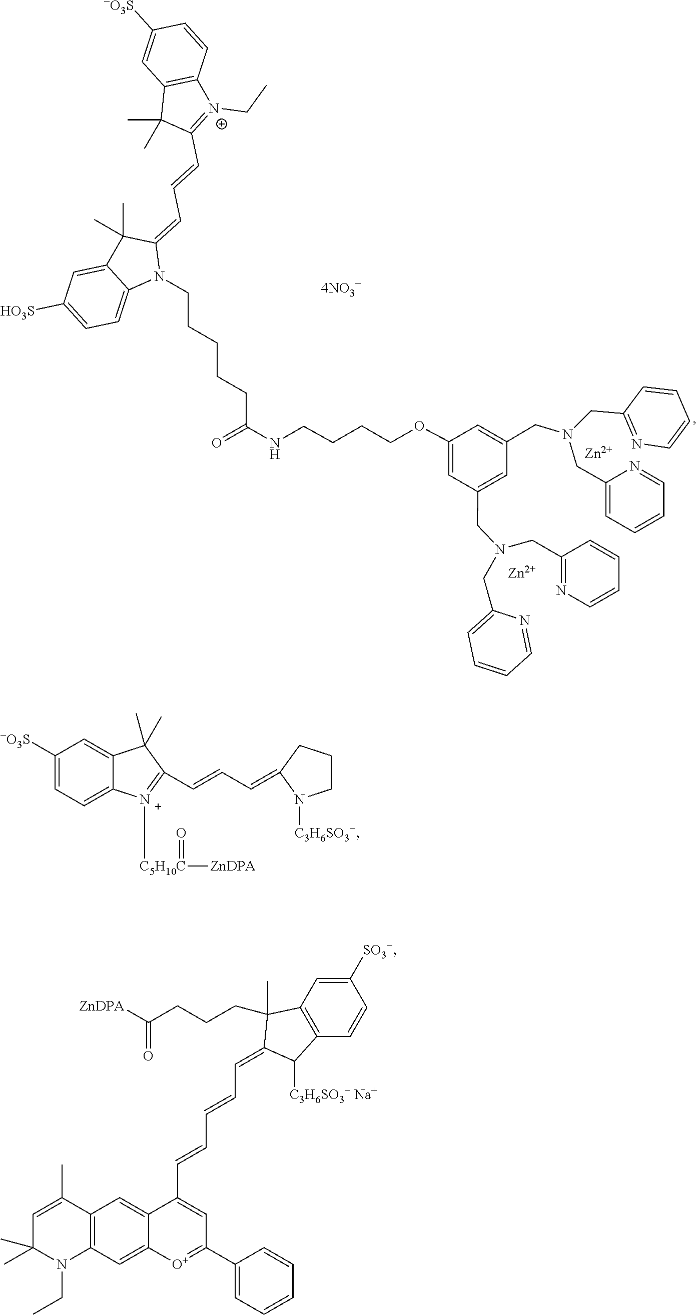 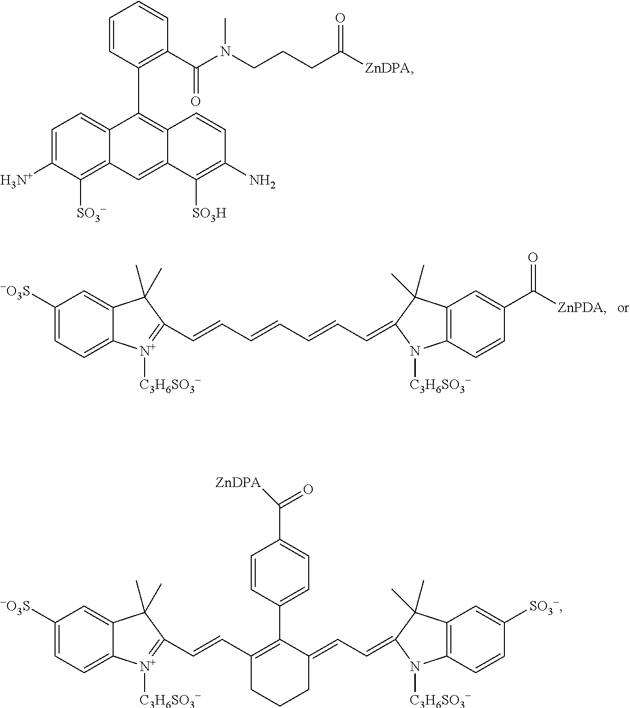
wherein ZnDPA has the following structure:

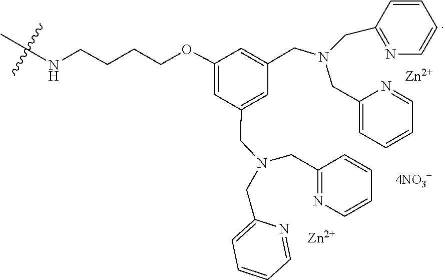 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

8. A method of determining the presence of and/or the amount of retinal apoptosis in an individual in need of treatment, comprising:
i) administering to an eye of the individual an effective amount of a first composition of claim 6; a second composition comprising a pharmaceutically acceptable carrier and a compound having the following structure: 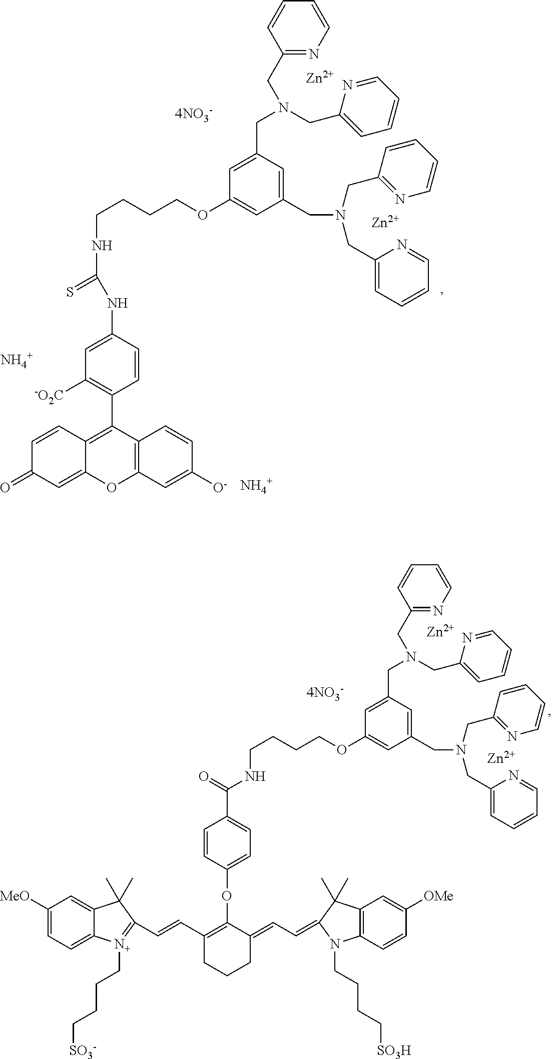 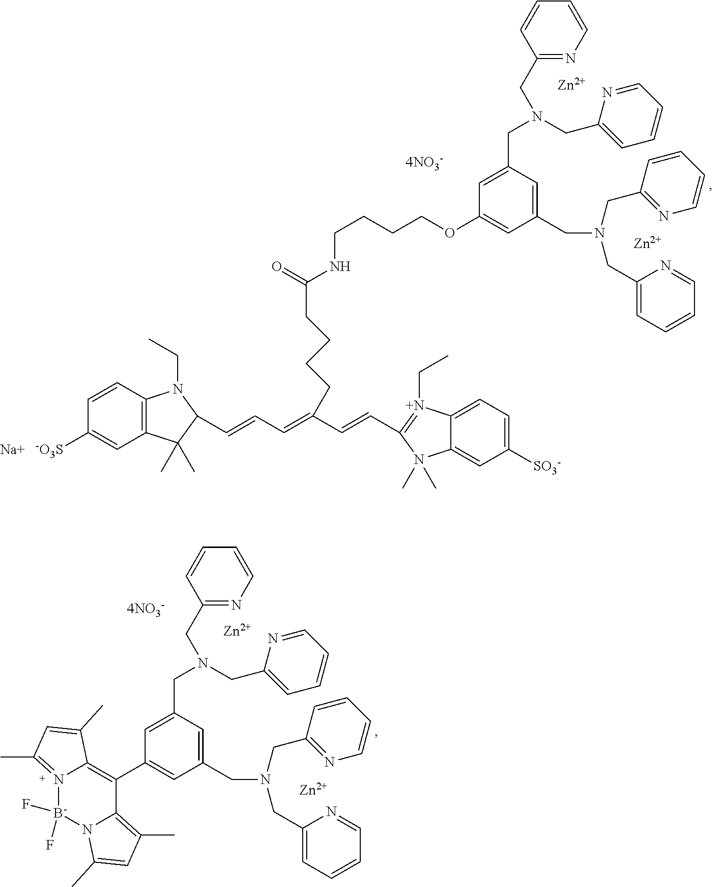 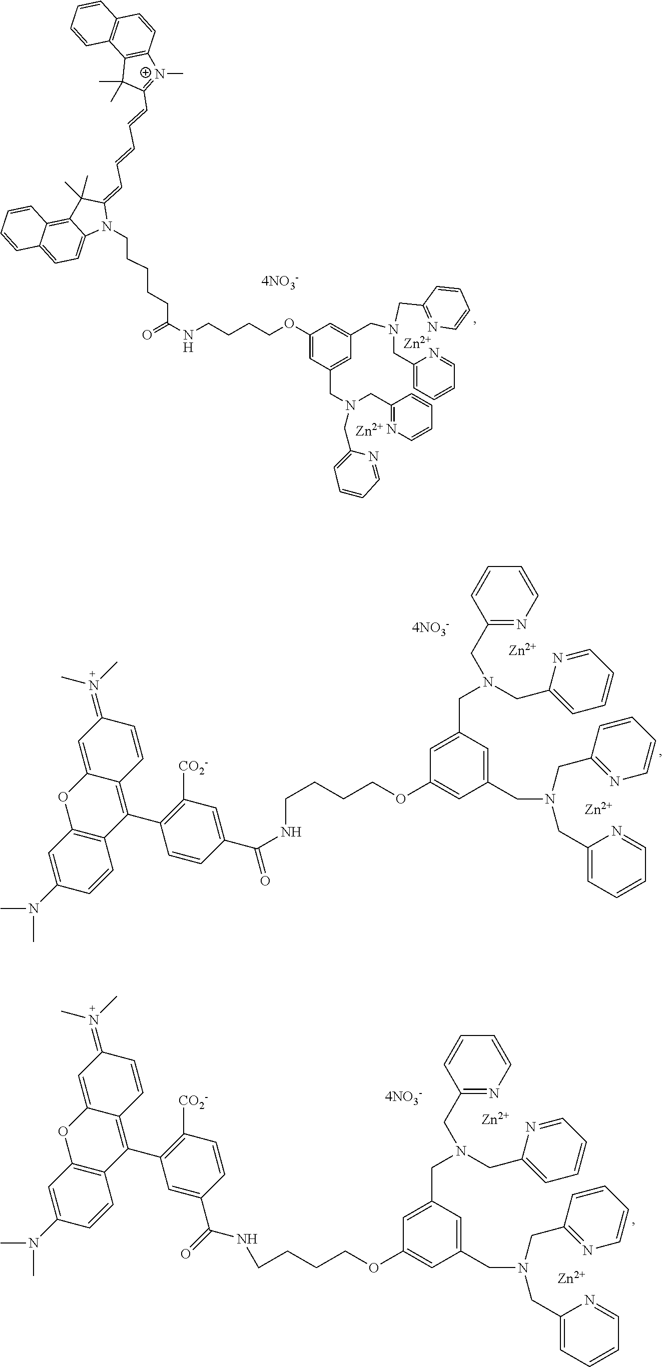
or a combination thereof, or a combination of the first composition and the second composition;
ii) dilating the pupil of the eye of the individual; and
iii) imaging the eye of the individual,
wherein the administration is not via intraocular injection.

9. The method of claim 8, wherein the first composition and/or second composition are administered as an eye drop.

10. The method of claim 9, wherein the compound(s) of the first composition and/or second composition have a concentration of 0.1-15 mM.

11. The method of claim 8, wherein the imaging comprises imaging via a retinal imaging system.

12. The method of claim 8, wherein the imaging comprises excitation with electromagnetic radiation.

13. The method of claim 12, wherein fluorescence emission is observed and occurs in the range of 440-900 nm.

14. The method of claim 13, wherein the fluorescence emission is quantified.

15. The method of claim 8, wherein the imaging is performed at up to 50 hours after administration.

16. The method of claim 8, wherein the individual in need of treatment has retinitis pigmentosa, primary open angle glaucoma, acute closed angle glaucoma, chronic closed angle glaucoma, myopic retinopathies, macular edema, genetic disease of the retina and macular, pars planitis, Posner Schlossman syndrome, Bechet's disease, Vogt-Koyanagi-Harada syndrome, hypersensitivity reactions, toxoplasmosis chorioretinitis, inflammatory pseudo-tumor of the orbit, chemosis, conjunctival venous congestion, periorbital cellulitis, acute dacryocystitis, nonspecific vasculitis, sarcoidosis, cytomegalovirus infection, diabetic retinopathy, age-related macular degeneration, or a combination thereof.

17. The method of claim 8, wherein the first composition and/or second composition are not detectable after 72 hours.

18. The method of claim 8, wherein the composition comprises: 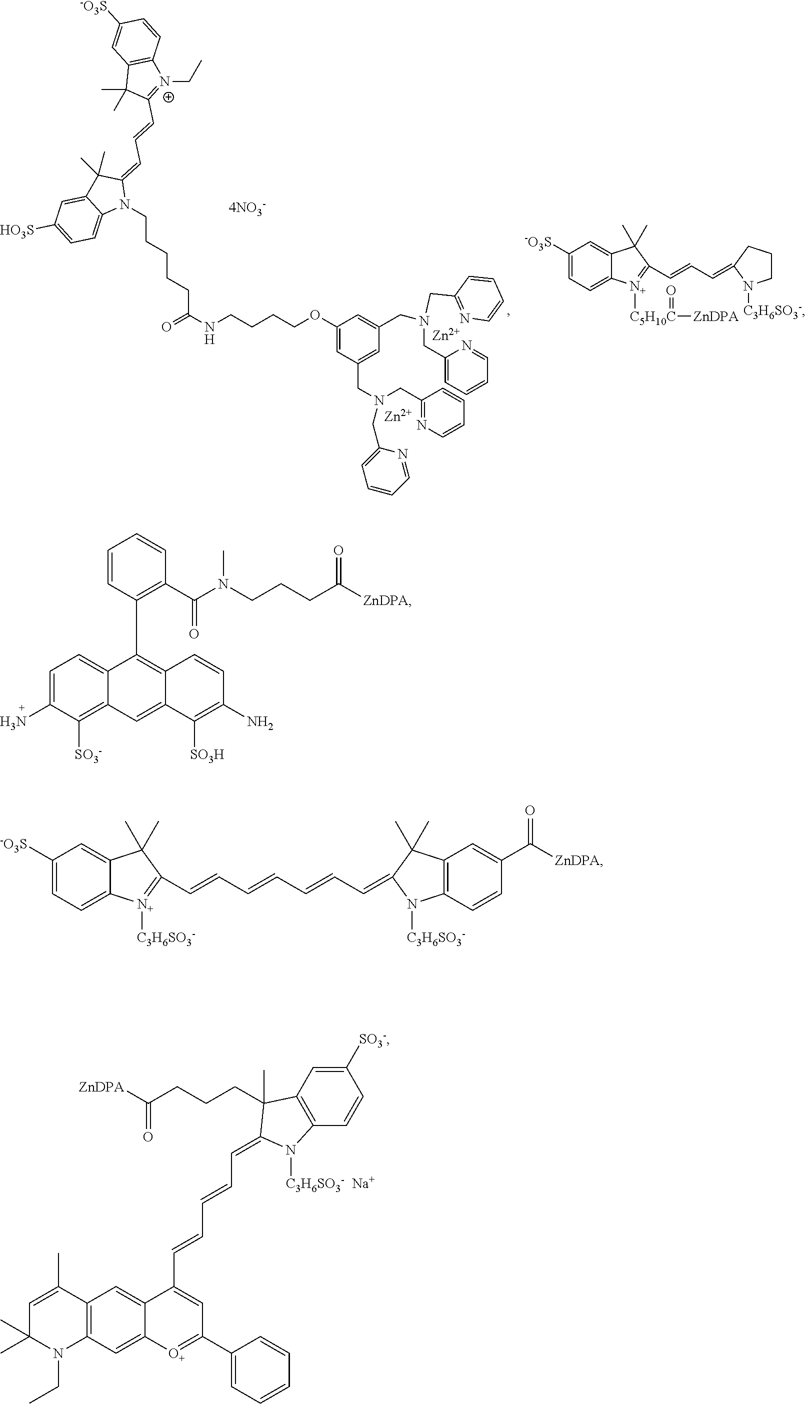 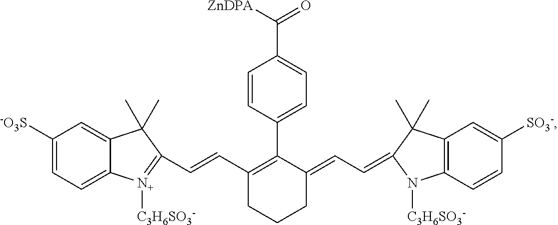
or a combination thereof
wherein ZnDPA has the following structure:

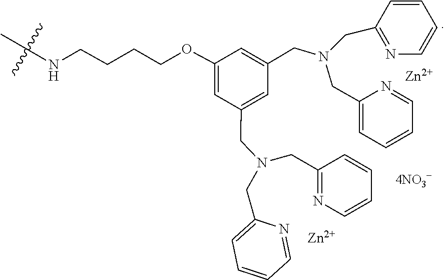 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

19. The method of claim 8, wherein the composition comprises: 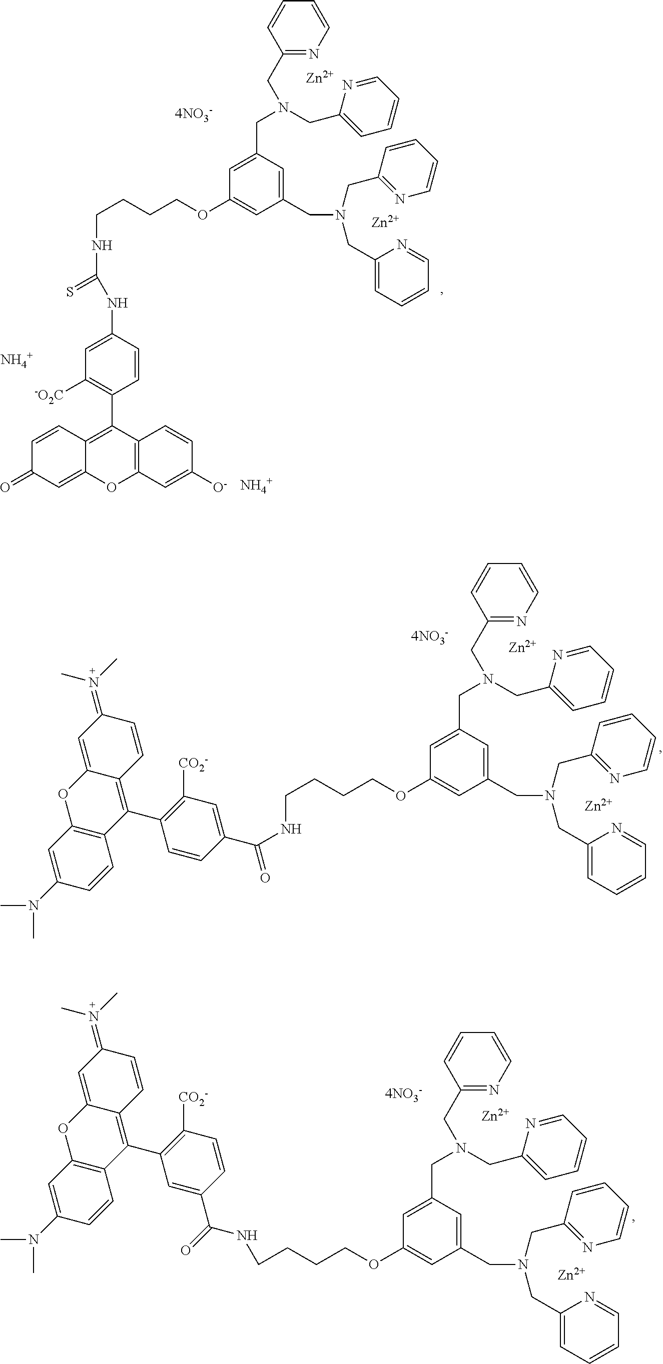 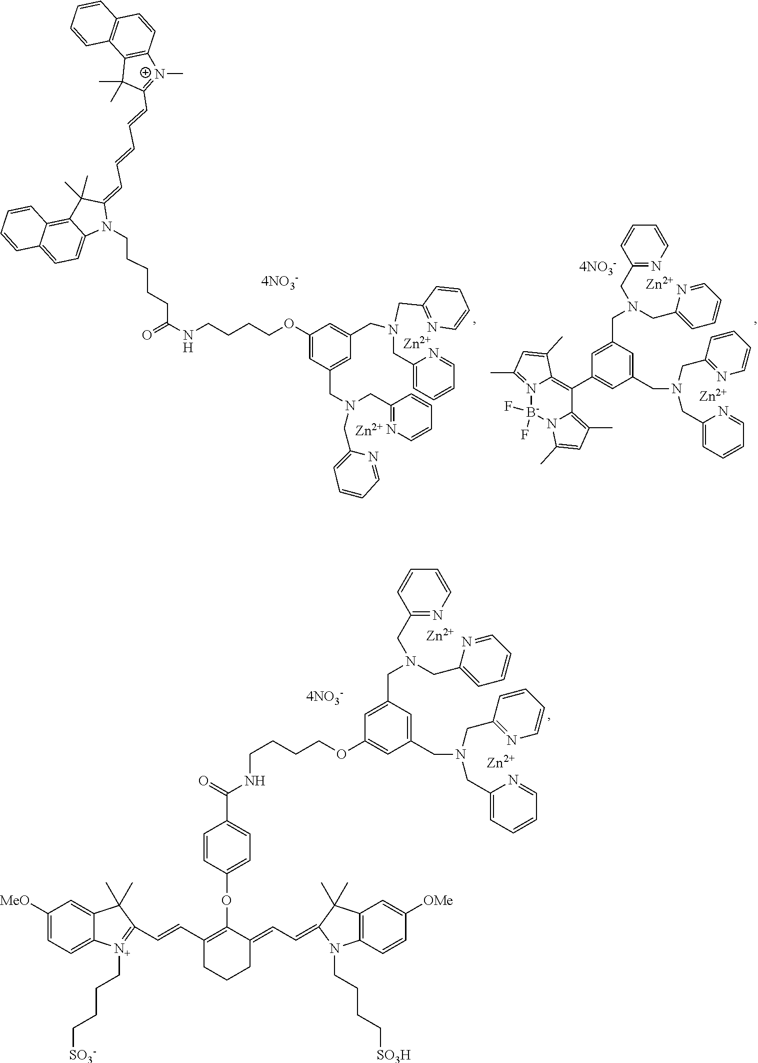
or a combination thereof.

20. An eye drop comprising a compound of claim 1.

21. The eye drop of claim 20, wherein the compound is chosen from: 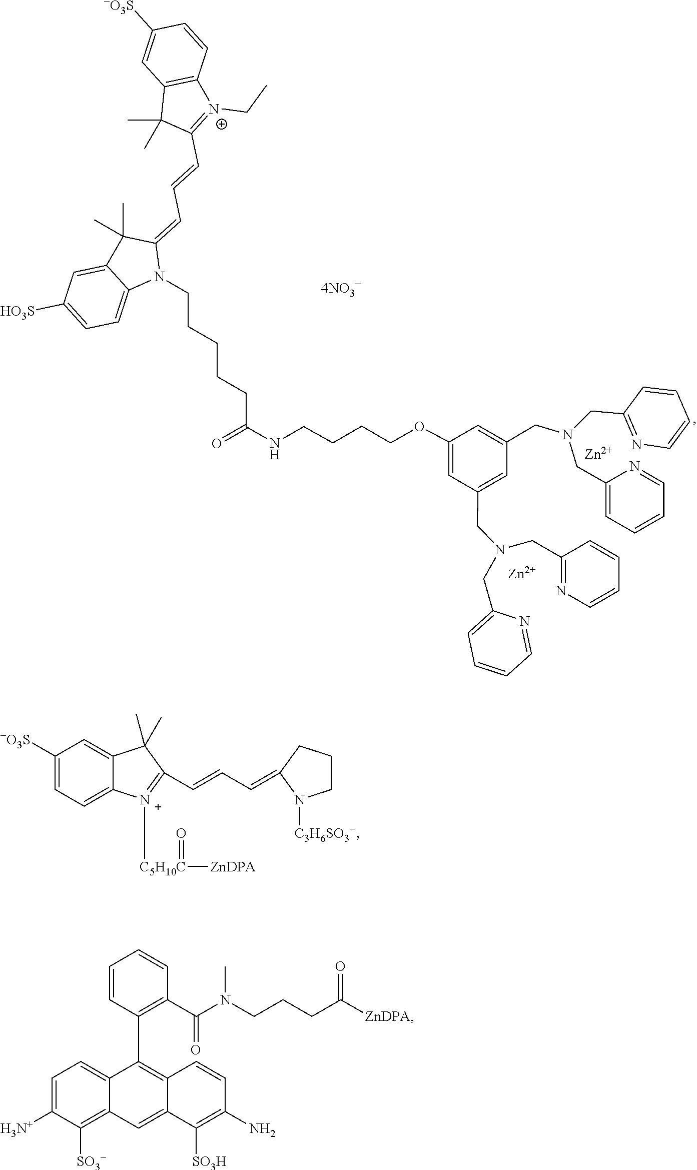 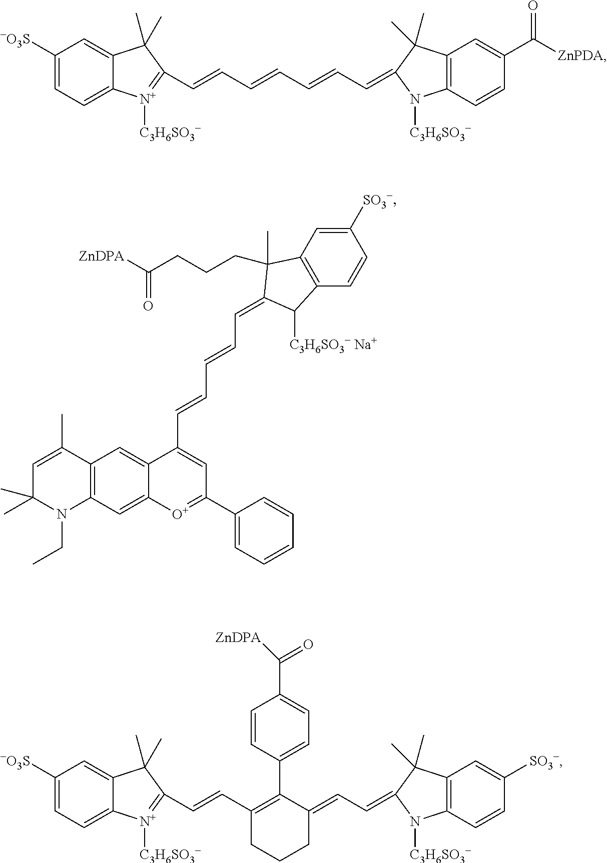
and combinations thereof,
wherein ZnDPA has the following structure:

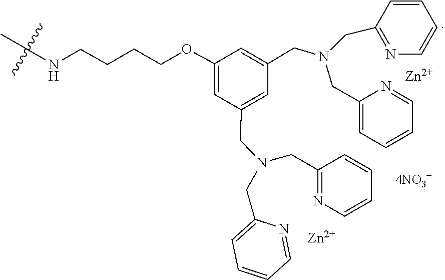 [SMILES: *NCCCCOc1cc(CN(Cc2ccccn2)Cc2ccccn2)cc(CN(Cc2ccccn2)Cc2ccccn2)c1 ; [Zn+2] ; [Zn+2]]

22. A method of determining effectiveness of a treatment for eye injury comprising
i) administering an eye drop of claim 20 to an eye of an individual,
ii) dilating the eye of the individual,
iii) imaging the eye of the individual,
iv) waiting a period of time,
v) optionally repeating steps i) and ii) or ii),
vi) imaging the eye of the individual, and
vii) comparing the images obtained from imaging,
wherein the comparison is used to the effectiveness of the treatment.
